(19) United States
(12) Patent Application Publication
(10) Pub. No.: US 2015/0105343 A1
(43) Pub. Date: Apr. 16, 2015

(54) NUCLEOSIDE SUPPLEMENTATION TO PROMOTE CELLULAR FUNCTION, GENETIC STABILITY AND REGENERATIVE APPLICATIONS

(71) Applicant: The Regents of the University of California, Oakland, CA (US)

(72) Inventors: James A. Byrne, Santa Monica, CA (US); Caius Radu, Los Angeles, CA (US); Patrick Lee, Los Angeles, CA (US)

(21) Appl. No.: 14/516,451

(22) Filed: Oct. 16, 2014

Related U.S. Application Data
(60) Provisional application No. 61/891,846, filed on Oct. 16, 2013.

(51) Int. Cl. C12N 5/074; A61K 31/7064

(52) U.S. Cl. CPC C12N 5/0696; A61K 31/7064; C12N 2500/42; C12N 2500/98; C12N 2500/92
USPC 514/52; 435/375

(57) ABSTRACT

In various embodiments, a cell culture medium, or a nucleoside cocktail transmission (NCT) medium for the culture of stem cells with improved genetic stability, cellular function and regeneration ability is provided. Illustrative culture media comprise a basal culture medium for stem cells, where the culture medium is supplemented with one or more nucleoside triphosphates (e.g., dNTPs and/or NPs) or one or more precursors thereof. Illustrative NCT media comprise a delivery vehicle (e g. skin creams or other vehicle) containing one or more nucleoside triphosphates (e.g., dNTPs and/or NPs) or one or more precursors thereof. The NCT medium provides, inter alia, direct delivery of nucleoside cocktails into human tissues (such as skin) for regenerative, cosmetic and/or therapeutic purposes.

### CROSS-REFERENCE TO RELATED APPLICATIONS

This application claims benefit of and priority to U.S. Ser. No. 61/891,846, filed on Oct. 16, 2013, which is incorporated herein by reference in its entirety for all purposes.

### STATEMENT OF GOVERNMENTAL SUPPORT

### Not Applicable

### BACKGROUND

Although induced pluripotent stem cells (iPSCs) share great similarities with embryonic stem cells (ESCs), the somatic cell reprogramming methods used for iPSC derivation have caused both scientific interest and concerns about the new cell type. There are important biosafety concerns about the genomic integrity and stability of iPSCs, as well as ESCs, during their derivation and prolonged culture, in addition to significant concerns regarding substandard/subphysiological cellular functions (such as suboptimal proliferation and differentiation) in suboptimally supplemented environmental conditions. The usefulness of human induced pluripotent stem cells (or other stem or somatic cells) in research, cosmetic and therapeutic/regenerative applications highly relies on their genomic integrity, stability and functionality. Certain genomic or epigenomic abnormalities may not only compromise the differentiation potential but also cause tumorigenesis in the recipients of iPSC-based therapies. Genomic abnormalities have been observed as karyotypic aberrations such as changes in chromosomal number and structures, copy number variations (CNVs) such as subkaryotypic or subchromosomal amplifications and deletions, loss of heterozygosity (LOH) due to acquired uniparental disomy, and random or site-specific integration of alien DNA into the host genome.

Studies indicate that various somatic and stem cells and essentially all IPSC clones derived using current approaches acquire genomic instability during reprogramming and in vitro culture-induced stress (Martins-Taylor and Xu (2012) Stem Cells 30: 22-27; Lund et al. (2012) Nat. Rev. Genet., 13: 732-744) in addition to possessing substandard physiology (such as impaired cellular proliferation). The issue of hiPSC genomic instability is one of the most important bottlenecks in advancing personalized iPSC-based regenerative therapies to the clinic (Martins-Taylor and Xu (2012) Stem Cells 30: 22-27; Lund et al. (2012) Nat. Rev. Genet., 13: 732-744; Byrne (2013) Gene Therapy and Regulation 7: 1230002), given the established link between genomic instability and an increased risk of malignant transformation.

The causes of genomic instability and impaired cellular physiology during hiPSC reprograming and culture, and indeed for other pluripotent stem cells (such as human embryonic stem cells) and somatic stem cells (such as neural stem cells, mesenchymal stem cells and dermal stem cells) are unclear, and approaches to reduce and eventually eliminate the occurrence of this highly deleterious phenomenon have yet to be developed.

### SUMMARY

It was discovered that the genomic integrity, stability and functionality, of stem cells and somatic cells can be augmented by exogenous supplementation of nucleosides (e.g., deoxyribonucleosides). Such a nucleoside supplement (refrred to as a nucloside cocktail (NC) or, in certain embodimetns a deoxyribonucleosideCocktail (DC) can be used to augment cell culture media or can be combined with a delivery vehicle to produce a nucleoside cocktail transmission (NCT) medium, or in certain embodiments a deoxyriobnucleoside transmission cocktail (DCT). Such supplementation, in addition to improving genomic stability can enhance cellular function (for example, by speeding up cell proliferation, promoting differentiation into target therapeutic cell types (such as hepatocytes), and/or promoting human tissue regeneration (such as stimulating division of human skin cells in vivo for regenerative purposes).

Moreover it was observed that exposure of human skin fibroblasts to a nucleoside cocktail can stimulate proliferation at a significantly augmented rate, and this phenomenon, when combined with nucleoside transmission media such as a skin cream, can prove very useful in directly regenerating/rejuvenating human skin or other tissues.

The nucleoside cocktail not only improves genomic stability, it also significantly augments proliferation rate of a wide variety of somatic and stem cells in addition to augmenting differentiation of pluripotent stem cells into target cell types (such as hepatocytes for treatment of liver disease).

Thus, some embodiments provide a basal culture medium for somatic and stem cells, where the culture medium is supplemented with one or more nucleoside triphosphates (e.g., dNTPs and/or NTPs) or one or more precursors thereof. Other embodiments combine the one or more nucleoside triphosphates (e.g., dNTPs and/or NTPs) or one or more precursors thereof with topical and/or delivery mechanisms in vivo use inclusive of, but not limited to: 1) skin creams, 2) topical cosmetics and/or 3) extrinsic derivery mechanisms (EDMs), such as injectables, ingestion, inhalation or other.

Accordingly, in various aspects, the invention(s) contemplated herein may include, but need not be limited to, any one or more of the following embodiments:

In various aspects, the invention(s) contemplated herein may include, but need not be limited to, any one or more of the following embodiments:

Embodiment 1: A cell culture medium for the culture of stem cells with improved genetic stability, said culture medium including: a basal culture medium for stem cells, where said culture medium is supplemented with one or more nucleoside triphosphates or one or more precursors thereof

Embodiment 2: A nucleoside cocktail transmission (NCT) medium for improving somatic or stem cell genetic stability said medium including: a cosmetic or pharmaceutical vehicle; and one or more nucleoside triphosphates or precursors thereof.

Embodiment 3: The cell culture medium of embodiment 1 or nucleoside cocktail transmission medium of embodiment 2, wherein said one or more nucleoside triphosphates are independently selected from the group consisting of deoxyadenosine triphosphate (dATP), deoxyguanosine triphosphate (dGTP), deoxycytidine triphosphate (NCTP), deoxythymidine triphosphate (dTTP) deoxyuridine triphosphate, adenosine triphosphate (ATP), guanosine triphosphate (GTP), cytidine triphosphate (CTP), 5-methyluridine triphosphate (m5UTP), and uridine triphosphate (UTP).

Embodiment 4: The cell culture medium of embodiment 1 or nucleoside cocktail transmission medium of embodiment 2, wherein said one or more nucleoside triphosphates are independently selected from the group consisting of deoxyadenosine triphosphate (dATP), deoxyguanosine triphosphate (dGTP), deoxycytidine triphosphate (NCTP), deoxythymidine triphosphate (dTTP) and deoxyuridine triphosphate.

Embodiment 5: The cell culture medium of embodiment 1 or nucleoside cocktail transmission medium of embodiment 2, wherein said one or more nucleoside triphosphates are independently selected from the group consisting of adenosine triphosphate (ATP), guanosine triphosphate (GTP), cytidine triphosphate (CTP), 5-methyluridine triphosphate (m5UTP), and uridine triphosphate (UTP).

Embodiment 6: The cell culture medium according to any one of embodiments 1 and 3-5 or nucleoside cocktail transmission medium according to any one of embodiments 2-5, wherein said culture medium is supplemented with, or said NCT medium includes, one or more precursors of nucleoside triphosphates independently selected from the group consisting of deoxyadenosine triphosphate (dATP), deoxyguanosine triphosphate (dGTP), deoxycytidine triphosphate (NCTP), deoxythymidine triphosphate (dTTP) deoxyuridine triphosphate, adenosine triphosphate (ATP), guanosine triphosphate (GTP), cytidine triphosphate (CTP), 5-methyluridine triphosphate (m5UTP), and uridine triphosphate (UTP).

Embodiment 7: The cell culture or NCT medium of embodiment 6, wherein said one or more precursors of nucleoside triphosphates are independently selected from the group consisting of deoxyadenosine triphosphate (dATP), deoxyguanosine triphosphate (dGTP), deoxycytidine triphosphate (NCTP), deoxythymidine triphosphate (dTTP) and deoxyuridine triphosphate.

Embodiment 8: The cell culture or NCT medium of embodiment 6, wherein said one or more precursors of nucleoside triphosphates are independently selected from the group consisting of adenosine triphosphate (ATP), guanosine triphosphate (GTP), cytidine triphosphate (CTP), 5-methyluridine triphosphate (m5UTP), and uridine triphosphate (UTP).

Embodiment 9: The cell culture medium according to any one of embodiments 1 and 3-8, or the NCT medium according to any one of embodiments 3-8, wherein said culture medium is supplemented with, or said NCT medium includes, deoxyadenosine triphosphate (dATP), or a precursor thereof

Embodiment 10: The cell culture medium or NCT medium of embodiment 9, said culture medium is supplemented with, or said NCT medium includes, deoxyadenosine triphosphate.

Embodiment 11: The cell culture medium or NCT medium of embodiment 9, said culture medium is supplemented with, or said NCT medium includes, a precursor of deoxyadenosine triphosphate.

Embodiment 12: The cell culture medium or NCT medium of embodiment 11, wherein said precursor of deoxyadenosine triphosphate is selected from the group consisting of deoxyadenosine diphosphate, deoxyadenosine monophosphate, deoxyadenosine, and adenine.

Embodiment 13: The cell culture medium according to any one of embodiments 1, and 3-12, or the NCT medium according to any one of embodiments 2-12, wherein said culture medium is supplemented with, or said NCT medium includes, deoxyguanosine triphosphate (dGTP), or a precursor thereof

Embodiment 14: The cell culture medium or NCT medium according of embodiment 13, wherein said cell culture medium is supplemented with or said NCT medium includes deoxyguanosine triphosphate.

Embodiment 15: The cell culture medium or NCT medium according of embodiment 13, wherein said cell culture medium is supplemented with or said NCT medium includes a precursor of deoxyguanosine triphosphate.

Embodiment 16: The cell culture or NCT medium of embodiment 15, wherein said precursor of deoxyguanosine triphosphate selected from the group consisting of deoxyguanosine diphosphate, deoxyguanosine monophosphate, deoxyguanosine, and guanine

Embodiment 17: The cell culture medium according to any one of embodiments 1, or 3-16, or the NCT medium according to any one of embodiments 2-16, wherein said culture medium is supplemented with or said NCT medium includes deoxycytidine triphosphate (NCTP), or a precursor thereof.

Embodiment 18: The cell culture medium or NCT medium of embodiment 17, wherein said cell culture medium is supplemented with, or said NCT medium includes deoxycytidine triphosphate.

Embodiment 19: The cell culture medium or NCT medium of embodiment 17, wherein said cell culture medium is supplemented with, or said NCT medium includes a precursor of deoxycytidine triphosphate.

Embodiment 20: The cell culture medium or NCT medium of embodiment 19, wherein precursor of deoxycytidine is selected from the group consisting of deoxycytidine diphosphate, deoxycytidine monophosphate, deoxycytidine, and cytosine.

Embodiment 21: The cell culture medium according to any one of embodiments 1, and 3-20, or the NCT medium according to any one of embodiments 2-20, wherein said culture medium is supplemented with, or said NCT includes deoxythymidine triphosphate (dTTP), or a precursor thereof.

Embodiment 22: The cell culture medium or NCT medium of embodiment 20, wherein said culture medium is supplemented with, or said NCT medium includes deoxythymidine triphosphate.

Embodiment 23: The cell culture medium or NCT medium of embodiment 20, wherein said culture medium is supplemented with, or said NCT medium includes a precursor of deoxythymidine triphosphate.

Embodiment 24: The cell culture medium or NCT medium of embodiment 23, wherein said precursor of deoxythymidine triphosphate is selected from the group consisting of deoxythymidine diphosphate, deoxythymidine monophosphate, deoxythymidine, and thymine.

Embodiment 25: The cell culture medium according to any one of embodiments 1, and 3-24, or the NCT medium according to any one of embodiments 2-24, wherein said culture or NCT medium is supplemented with deoxyuridine triphosphate, or a precursor thereof

Embodiment 26: The cell culture medium or NCT medium of embodiment 25, wherein said culture medium is supplemented with, or said NCT medium includes deoxyuridine triphosphate.

Embodiment 27: The cell culture medium or NCT medium of embodiment 25, wherein said culture medium is supplemented with, or said NCT medium includes a precursor of deoxyuridine triphosphate.

Embodiment 28: The cell culture medium or NCT medium of embodiment 27, wherein said precursor of deoxyuridine triphosphate is selected from the group consisting of deoxyuridine diphosphate, deoxyuridine monophosphate, deoxyuridine, and uracil.

Embodiment 29: The cell culture medium according to any one of embodiments 1, and 3-28, or the NCT medium according to any one of embodiments 2-28, wherein said culture or NCT medium is supplemented with adenosine triphosphate (ATP) or a precursor thereof

Embodiment 30: The cell culture medium or NCT medium of embodiment 29, wherein said culture medium is supplemented with, or said NCT medium includes, adenosine triphosphate (ATP).

Embodiment 31: The cell culture medium or NCT medium of embodiment 29, wherein said culture medium is supplemented with, or said NCT medium includes, a precursor of adenosine triphosphate.

Embodiment 32: The cell culture medium or NCT medium of embodiment 31, wherein said precursor of adenosine triphosphate (ATP) is selected from the group consisting of adenosine diphosphate, adenosine monophosphate, adenosine, and adenine.

Embodiment 33: The cell culture medium according to any one of embodiments 1, and 3-32, or the NCT medium according to any one of embodiments 2-32, wherein said culture medium is supplemented with, or said NCT medium includes guanosine triphosphate (GTP) or a precursor thereof.

Embodiment 34: The cell culture medium or NCT medium of embodiment 33, wherein said culture medium is supplemented with, or said NCT medium includes, guanosine triphosphate (GTP).

Embodiment 35: The cell culture medium or NCT medium of embodiment 33, wherein said culture medium is supplemented with, or said NCT medium includes, a precursor of guanosine triphosphate.

Embodiment 36: The cell culture medium or NCT medium of embodiment 35, wherein said precursor of guanosine triphosphate is selected from the group consisting of guanosine diphosphate, guanosine monophosphate, guanosine, and guanine

Embodiment 37: The cell culture medium according to any one of embodiments 1, and 3-36, or the NCT medium according to any one of embodiments 2-36, wherein said cell culture medium is supplemented with, or said NCT medium includes cytidine triphosphate (CTP), or a precursor thereof

Embodiment 38: The cell culture medium or NCT medium of embodiment 37, wherein said culture medium is supplemented with, or said NCT medium includes, cytidine triphosphate.

Embodiment 39: The cell culture medium or NCT medium of embodiment 37, wherein said culture medium is supplemented with, or said NCT medium includes, a precursor of cytidine triphosphate.

Embodiment 40: The cell culture medium or NCT medium of embodiment 39, wherein said precursor of cytidine triphosphate is selected from the group consisting of cytidine diphosphate, cytidine monophosphate, cytidine, and cytosine.

Embodiment 41: The cell culture medium according to any one of embodiments 1, and 3-40, or the NCT medium according to any one of embodiments 2-40, wherein said culture or NCT medium is supplemented with 5-methyluridine triphosphate (m5UTP), or a precursor thereof.

Embodiment 42: The cell culture medium or NCT medium of embodiment 41, wherein said culture medium is supplemented with, or said NCT medium includes, 5-methyluridine triphosphate.

Embodiment 43: The cell culture medium or NCT medium of embodiment 41, wherein said culture medium is supplemented with, or said NCT medium includes, a precursor of 5-methyluridine triphosphate.

Embodiment 44: The cell culture medium or NCT medium of embodiment 43, wherein said precursor of 5-methyluridine triphosphate is selected from the group consisting of 5-methyluridine diphosphate, 5-methyluridine monophosphate, 5-methyluridine, and thymine.

Embodiment 45: The cell culture medium according to any one of embodiments 1, and 3-44, or the NCT medium according to any one of embodiments 2-44, wherein said culture medium is supplemented with, or said NCT medium includes uridine triphosphate (UTP), or a precursor thereof

Embodiment 46: The cell culture medium or NCT medium of embodiment 45, wherein said culture medium is supplemented with, or said NCT medium includes uridine triphosphate.

Embodiment 47: The cell culture medium or NCT medium of embodiment 45, wherein said culture medium is supplemented with, or said NCT medium includes a precursor of uridine triphosphate.

Embodiment 48: The cell culture medium or NCT medium of embodiment 47, wherein precursor of uridine triphosphate is selected from the group consisting of uridine diphosphate, uridine monophosphate, uridine, and uracil.

Embodiment 49: The nucleoside cocktail transmission (NCT) medium according to any one of embodiments 2-48, wherein said NCT medium includes a vehicle formulated for administration via a route selected from the group consisting of subcutaneous administration, parenteral administration, topical administration, oral administration, nasal or inhalation administration, local administration such as by paint, aerosol, or transdermally.

Embodiment 50: The nucleoside cocktail transmission (NCT) medium according to any one of embodiments 2-48, wherein said NCT medium includes a vehicle formulated for topical administration, subdermal administration, or intradermal administration.

Embodiment 51: The nucleoside cocktail transmission (NCT) medium of embodiment 50, wherein said medium is formulated in a vehicle selected from the group consisting of a mud, an herbal mixture, a fat, an emulsions, a lotion, a cream, a gel, a biological, a solution, a spray, an ointment, a foams, a mousses, a liquid, a suspensions, a dispersion, an aerosol, a soap, a shampoo, and a conditioner.

Embodiment 52: The nucleoside cocktail transmission (NCT) medium of embodiment 50, wherein said medium is formulated as a cream (e.g., a face cream).

Embodiment 53: The nucleoside cocktail transmission (NCT) medium according to any one of embodiments 56-58, wherein said medium includes a formulation selected from the group consisting of a wrinkle removing cream, a dermal filler, a scar-reducing cream, and an acne treatment.

Embodiment 54: The cell culture medium according to any one of embodiments 1, and 3-48 or the nucleoside cocktail transmission (NCT) medium according to any one of embodiments 2-53, wherein said nucleoside triphosphate or precursor thereof supplementing said culture medium, or including said NCT medium, are present at a concentration sufficient to improve the short term and/or long term genetic stability of stem cells as compared to the same cells cultured in the same medium without supplementation by a nucleoside triphosphate or precursor thereof

Embodiment 55: The cell culture medium or the nucleoside cocktail transmission (NCT) medium of embodiment 54, wherein said stem cells are stem cells selected from the group consisting of somatic cells (e.g. fibroblasts) or stem cells (e.g., neural stem cells, mesenchymal stem cells, hematopoietic stem cells, adipose stem cells, embryonic stem cells, cord stem cells, and induced pluripotent stem cells).

Embodiment 56: The cell culture medium according to any one of embodiments 1, 3-48, and 54-55, or the NCT medium according to any one of embodiments 2-55, wherein each nucleoside triphosphate or precursor thereof supplementing said culture medium, or including said NCT medium is present at a concentration (e.g., either in transmission medium and/or resulting in such a concentration on target cells in vivo, such as extrinsic delivery of nucleoside cocktail into human skin using a skin cream as the NCT medium) ranging from about 1 μM up to about 50 μM, or from about 1 μM to about 40 μM, or from about 1 μM up to about 35 μM, or from about 1 μM up to about 30 μM.

Embodiment 57: The cell culture medium according to any one of embodiments 1, 3-48, and 54-55, or the NCT medium according to any one of embodiments 2-55, wherein each nucleoside triphosphate or precursor thereof supplementing said culture medium, or included in said NCT medium, is at a concentration starting (stock) or final (extrinsic delivery) of about 50 μM or lower, or about 40 μM or lower, or about 30 μM or lower or about 25 μM or lower, or about 20 μM or lower, or about 15 μM or lower, or about 10 μM or lower, or about 5 μM or lower.

Embodiment 58: The cell culture medium, or NCT medium of embodiment 57, wherein each nucleoside triphosphate or precursor thereof supplementing said culture medium, or included in said NCT medium, is present at a starting (stock) or final (extrinsic delivery) concentration ranging from about 5₁LIM to about 30 _{I}LIM.

Embodiment 59: The cell culture medium, or NCT medium of embodiment 57, wherein each nucleoside triphosphate or precursor thereof supplementing said culture said culture medium, or included in said NCT medium, is present at a starting (stock) or final (extrinsic delivery) concentration of about 30 μM.

Embodiment 60: The cell culture medium, or NCT medium of embodiment 57, wherein each nucleoside triphosphate or precursor thereof supplementing said culture said culture medium, or included said NCT medium, is present at a starting (stock) or final (extrinsic delivery) concentration of about 5 μM.

Embodiment 61: The cell culture medium according to any one of embodiments 1, 3-48, and 54-60, or the NCT medium according to any one of embodiments 2-60, wherein said cell culture medium, or said NCT medium is xenopathogen-free.

Embodiment 62: The cell culture medium according to any one of embodiments 1, 3-48, and 54-61, or the NCT medium according to any one of embodiments 2-61, wherein said cell culture medium, or said NCT medium is without animal or human derived serum albumin.

Embodiment 63: The cell culture medium according to any one of embodiments 1, 3-48, and 54-62, wherein the supplemented medium is selected from the group consisting of DMEM (Dulbecco's Modified Eagle's Medium), MEM (Minimal Essential Medium), BME (Basal Medium Eagle), RPMI 1640, DMEM/F-12 (Dulbecco's Modified Eagle's Medium: Nutrient Mixture F-12), DMEM/F-10 (Dulbecco's Modified Eagle's Medium: Nutrient Mixture F-10), α-MEM (α-Minimal essential Medium), G-MEM (Glasgow's Minimal Essential Medium), IMDM (Isocove's Modified Dulbecco's Medium), essential 8 (E8) medium, and KnockOut DMEM.

Embodiment 64: The cell culture medium of embodiment 63, wherein the basal medium is DMEM/F12.

Embodiment 65: The nucleoside cocktail transmission (NCT) medium of embodiment 2, wherein said NCT medium comprises said vehicle and a cell culture medium according to any one of embodiments 1, 3-48, and 55-64.

Embodiment 66: A somatic or stem cell culture or nucleoside cocktail transmission (NCT) culture, said cell culture including cells in a cell culture medium according to any one of embodiments 1, 3-48, and 55-64, or including cells in an NCT medium according to any one of embodiments 2-62, and 65.

Embodiment 67: The cell culture or NCT culture of embodiment 66, wherein said somatic or stem cells are cells selected from the group consisting of somatic cells (e.g., fibroblasts) or stem cells (e.g., neural stem cells, mesenchymal stem cells, hematopoietic stem cells, adipose stem cells, embryonic stem cells, cord stem cells, induced pluripotent stem cells, and the like).

Embodiment 68: The cell culture or NCT culture of embodiment 66, wherein said cells comprise stem cells.

Embodiment 69: The cell culture or NCT culture of embodiment 68, wherein said stem cells are selected from the group consisting of embryonic stem cells and adult stem cells, including, but not limited to neural stem cells, hepatic stem cells, hematopoietic stem cells, umbilical cord blood stem cells, epidermal stem cells, gastrointestinal stem cells, endothelial stem cells, muscle stem cells, mesenchymal stem cells, and pancreatic stem cells.

Embodiment 70: The cell culture or NCT culture of embodiment 68, wherein said cells are induced pluripotent stem cells (IPSCs) (e.g., such as autologous hIPSCs derived from a patient).

Embodiment 71: The cell culture or NCT culture of embodiment 70, wherein said IPSCs are reprogrammed from cells selected from the group consisting of fibroblasts, neural stem cells, stomach cells, liver cells, keratinocytes, melanocytes, amniotic cells, blood cells, 13-cells, and adipose cells.

Embodiment 72: The cell culture or NCT culture of embodiments 70 or 71, wherein the IPSCs comprise cells reprogramed two or more factors selected from the group consisting of KLF4 (K), LIN28 (L), c-MYC (M), NANOG (N); OCT4 (0), SOX2 (S), and valproic acid (VPA).

Embodiment 73: The cell culture or NCT culture of embodiment 72, wherein the ISPCs includes cells reprogramed using the four canonical Yamanaka factors KLF4 (K), c-MYC (M),OCT4 (O), and SOX2 (S).

Embodiment 74: The cell culture or NCT culture of embodiment 73, wherein the reprogramming factors further comprise LIN28.

Embodiment 75: The cell culture or NCT culture according to any one of embodiments 70-74, wherein said IPSCs are reprogrammed using a vector selected from the group consisting of an integrating vector, a non-integrating vector, an excisable vector, and a DNA-free vector.

Embodiment 76: A method of reducing genetic instability of stem cells said method including culturing said cells in a cell culture medium according to any one of embodiments of embodiments a culture medium according to any one of embodiments 1, 3-48, and 55-64.

Embodiment 77: A method of performing autologous stem cell transfer, said method including isolating stem cells from a subject or generating IPSCs from said subject and expanding and/or culturing said stem cells or IPSCs in a cell culture medium according to any one of embodiments 1, 3-48, and 55-64, or in an NCT medium according to any one of embodiments 2-62, and 65.

Embodiment 78: A method of promoting regeneration and/or maintenance of tissues, said method including administering to a subject, an NCT medium according to any one of embodiments 2-62, and 65.

Embodiment 79: The method of embodiment 78, wherein said NCT medium includes a vehicle formulated for administration via a route selected from the group consisting of subcutaneous administration, parenteral administration, topical administration, oral administration, nasal or inhalation administration, local administration such as by paint, aerosol, or transdermally.

Embodiment 80: The method of embodiment 78, wherein, wherein said NCT medium includes a vehicle formulated for topical administration, subdermal administration, or intradermal administration.

Embodiment 81: The method of embodiment 80, wherein said NCT medium is formulated in a vehicle selected from the group consisting of a mud, an herbal mixture, a fat, an emulsions, a lotion, a cream, a gel, a biological, a solution, a spray, an ointment, a foams, a mousses, a liquid, a suspensions, a dispersion, an aerosol, a soap, a shampoo, and a conditioner.

Embodiment 82: The method of embodiment 80, wherein said NCT medium is formulated as a cream (e.g., a face cream).

Embodiment 83: The method of embodiment 80, wherein said NCT medium includes a formulation selected from the group consisting of a wrinkle removing cream, a dermal filler, a scar-reducing cream, and an acne treatment.

Embodiment 84: The method according to any one of embodiments 78-83, wherein said NCT is applied to the skin of a human.

Embodiment 85: The method of embodiment 84, wherein said NCT is applied to reduce scarring.

Embodiment 86: The method of embodiment 84, wherein said NCT is applied to reduce wrinkles

Embodiment 87: The method according to any one of embodiments 78-83, wherein said NCT is applied intradermally or subdermally to increase tissue volume.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Characterization of human induced pluripotent stem cells (hIPSCs). (a) Morphology and immunocytochemical detection of stem cell markers (insert is DAPI); (b) Histological characterization following H&E staining of the three germ layers observed in transgene-free hIPSC-derived teratomas, (c) Karyotype analysis of the transgene-free iPSCs before and after CC-based stress. Red arrow highlights extra chromosome 12. Scale bar equals 100 microns.

FIG. 2: Significant reduction of dNTP pools following hIPSC reprogramming. *The size of the dNTP pool was standardized relative to the level observed in the original dermal fibroblasts.

FIG. 3A and 3B: Visualization (FIG. 3A) and quantification (FIG. 3B) of YH2A.X positive foci. Green staining highlights yH2A.X foci, blue staining highlights DAPI stained nuclei. HDFs were reprogrammed into hIPSCs and then cultured for four days with and without deoxyribonucleoside (dN) supplementation.

FIG. 4: Single nucleotide polymorphism (SNP)-based loss of heterozygosity (LOH) analysis following “clinical-grade conversion” (CC) based stress.

FIG. 5: Supplementing with 30 μM of dNs significantly increases hIPSC proliferation rate. First experiment treated cells with 0 μM, 30 μM, 100 μM, 200 μM dN over 5 day period. 200 μM was observed to be toxic (data not shown). Second experiment: treated cells with 0 μM, 30 μM, 50 μM, 75 μM, 100 μM dN over 5 day period

FIG. 6 illustrates a significant reduction of dNTP pools following hIPSC reprogramming and dNTP rescue with deoxyribonucleoside (dN) culture media supplementation. The size of the dNTP pool was standardized relative to the level observed in the original skin cells.

FIG. 7 shows visualization and quantification of γH2A.X positive foci (indicative of double strand breaks).

FIG. 8 shows that use of deoxyribonucleoside cocktail (DC) can stimulate both skin fibroblasts and human induced pluripotent stem cells to divide more quickly. Cell counts of fibroblasts and stem cells grown with and without supplement were taken at 0 and 48 hours post-plating. For the AD1 stem cells, one subclone that had been reprogrammed with culture supplement was taken off supplement temporarily to conduct doubling rate experiments. The equation used to calculate population doubling time (PDT) 48 your post plating was 48/(Log (T₄₈)-Log (Tₒ)). To calculate doubling rate per 24 hours: 24/PDT.

FIG. 9 shows that a nucleoside cocktail (NC) can significantly rescue dNTP pools in hIPSCs.

FIG. 10 shows visualization and quantification of γH2A.X positive foci (indicative of double strand breaks) reducing in the presence of a nucleoside coctail. The incidence of DNA damage (via γH2AX expression) was decreased with culture supplement. DNA damage levels were measured via γH2AX expression (green) which is indicative of double-stranded breaks. Blind counts were conducted to determine percent damaged nuclei. Supplement causes a decrease in damage in two separate stem cell lines.

FIG. 11 shows differentiation of pluripotent stem cells into a therapeutic target cell type (in this case hepatocyte-like cells, hereby called hepatocytes).

FIG. 12 shows alpha-fetoprotein expressing hepatocytes can be derived within two weeks when DC supplement used, but not without DC.

### DETAILED DESCRIPTION

In various embodiments, the methods an decompositions described herein pertain to the discovery that deoxyribonucleoside (dN) and/or nucleoside (ribonucleoside) supplementation can increase genetic stability in stem cells including induced pluripotent stem cells (IPSCs) in culture.

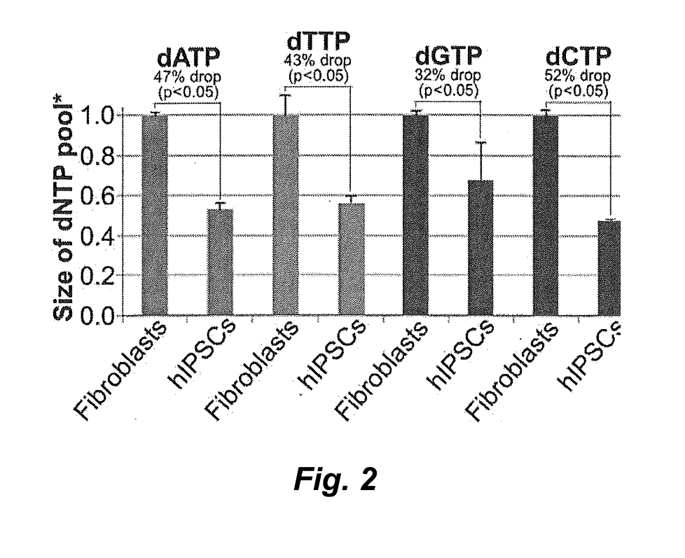

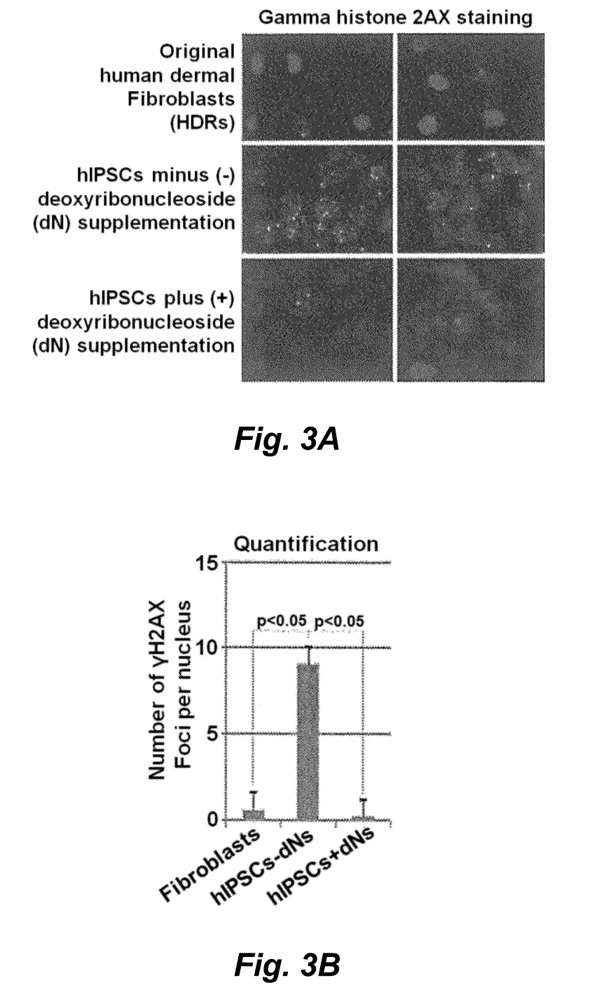

As demonstrated herein, it was determined that deoxyribonucleotide triphosphate (dNTP) pools in rapidly dividing hIPSCs are significantly smaller in size than the dNTP pools of cells (e.g., dermal fibroblasts, from which the hIPSCs are derived (see, e.g., FIGS. 2 and 6). It was also determined that hIPSCs demonstrate significantly more double strand breaks (DSBs) than the fibroblasts of origin (see, e.g., FIGS. 3A and 3B).

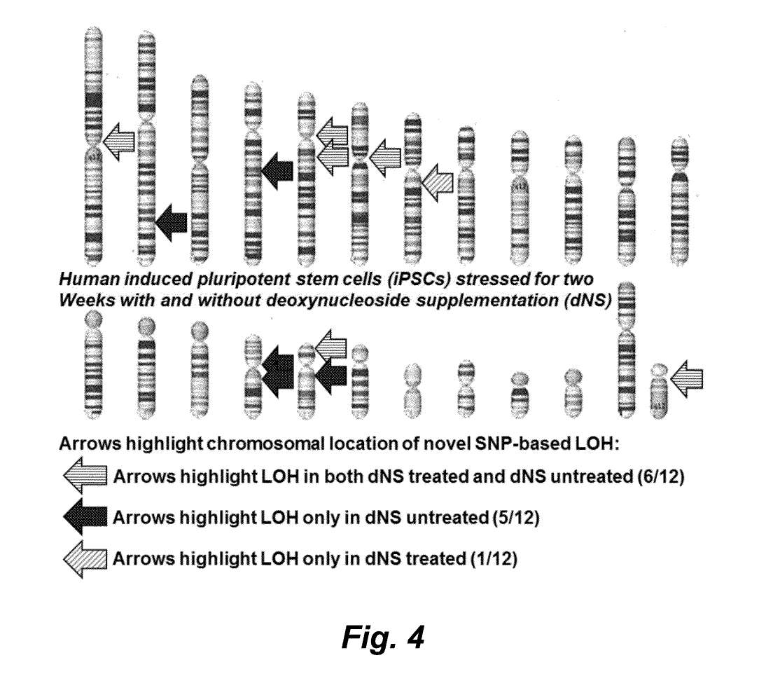

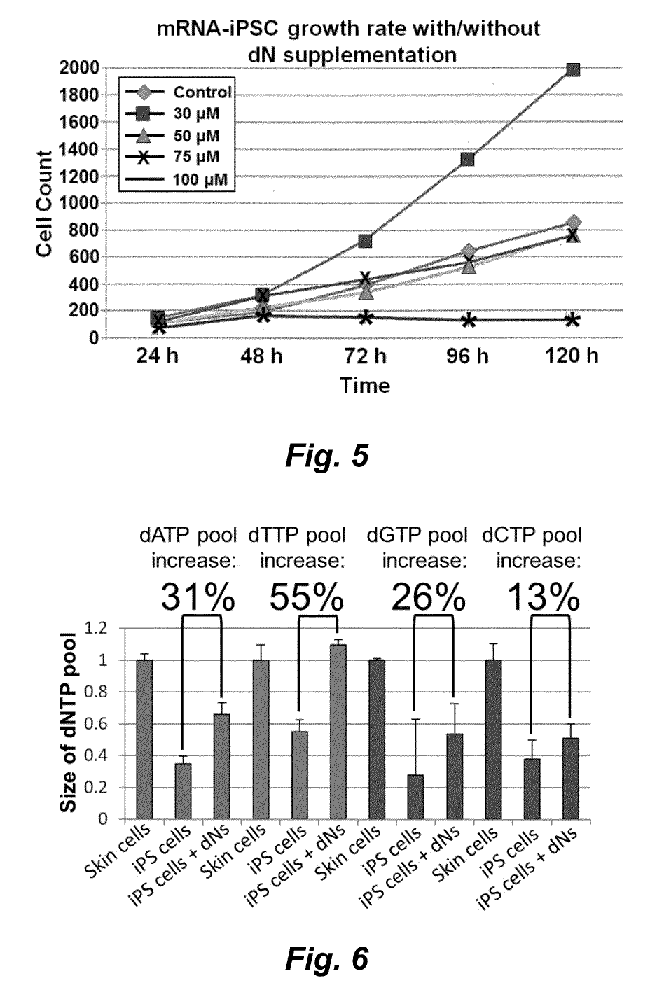

Data presented herein indicate that: 1) hIPSCs can utilize exogenous NTP precursors added to the hIPSC culture or DCT medium in the form of a mixture of deoxyribonucleosides (dNs) and 2) that culture medium supplementation with deoxyribonucleoside (dN) and/or nucleosides or precursors thereof can both ameliorate the hIPSC dNTP pool deficiency and significantly reduce the genomic instability experienced by these cells (see, e.g., FIG. 4).

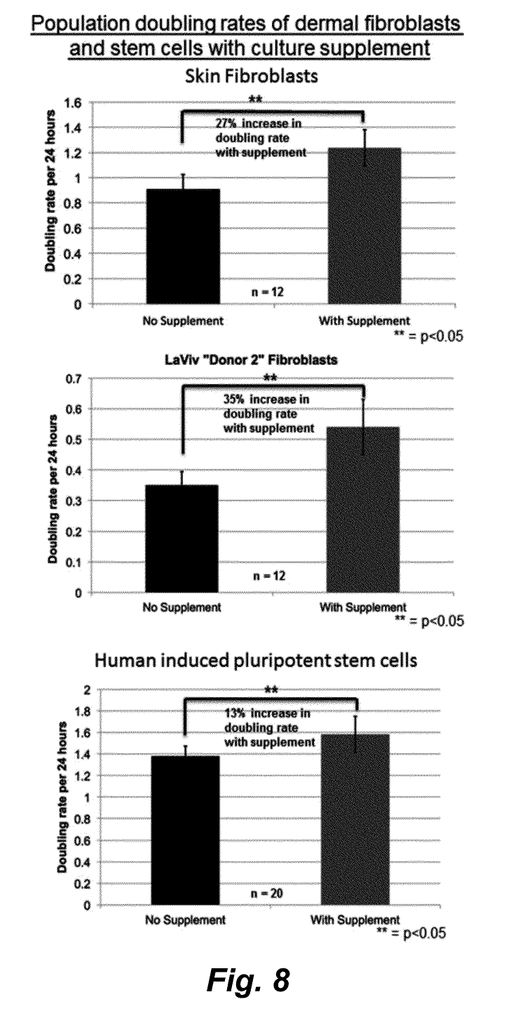

It was also discovered that exogenous NTP precursors can be incorporated into delivery vehicles (e.g., pharmaceutical delivery vehicles, cosmetic delivery vehicles, cosmetics, etc.) to form a nucleoside cocktail transmission (NCT) medium that can significantly stimulate proliferation of stimulate cellular proliferation of both human skin cells and human induced pluripotent stem cells (see, e.g., FIG. 8). As used here, the terms nucleoside cocktail transmission (NCT) or nucleoside cocktail delivery (NCD) medium refers to a formulation comprising a vehicle supplemented with nucleosides or precursors thereof or deoxynucleosides or precursors thereof formulated for administration in vivo to a subject (e.g., a human, a non-human mammal, etc.). Deoxynucleoside cocktail transmission (DCT) medium or deoxynucleoside cocktail delivery (DCD) medium refers to to a formulation comprising a vehicle supplemented with deoxynucleosides or precursors thereof formulated for administration in vivo to a subject (e.g., a human, a non-human mammal, etc.).

It is believed that a nucleoside cocktail can also stimulate cellular proliferation of other cells in vitro as well as stimulating cellular proliferation and/or tissue rejuvenation/regeneration in vivo (such as following direct topical application of an NCT in the form of, for example, a skin cream).

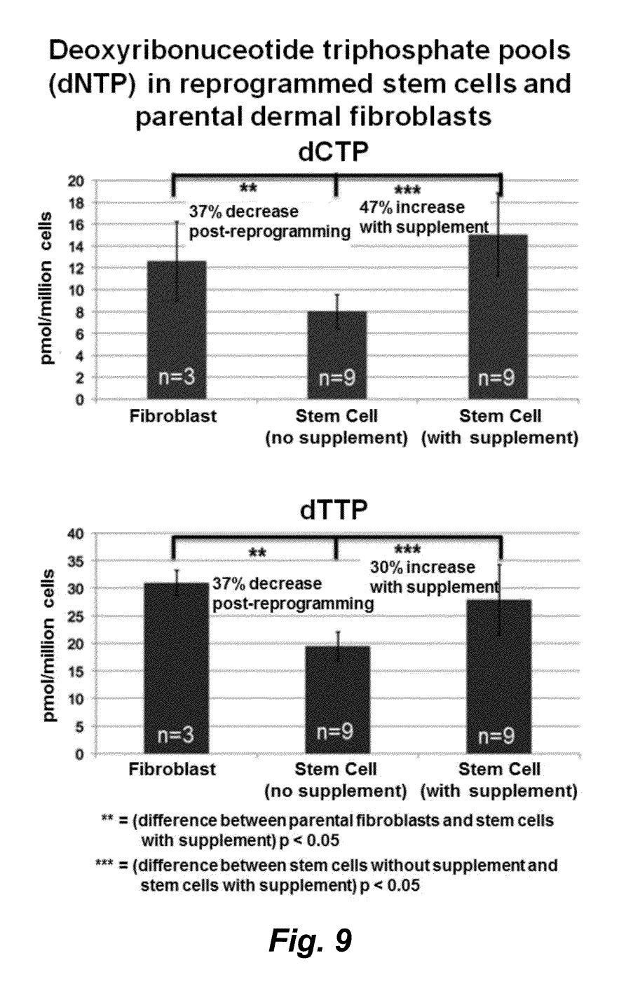

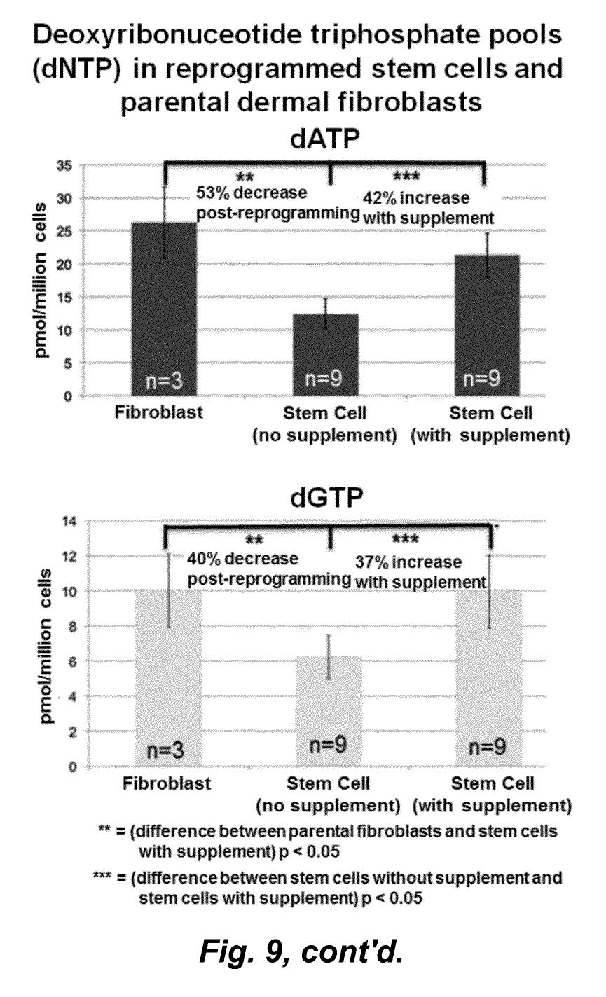

Data presented herein indicate that nucleoside supplementation can significantly rescue dNTP pools in human induced pluripotent stem cells (see, e.g., FIG. 9) and it is believed that nucleoside supplementation can also augment dNTP pool size in a wide range of other human and non-human cell types.

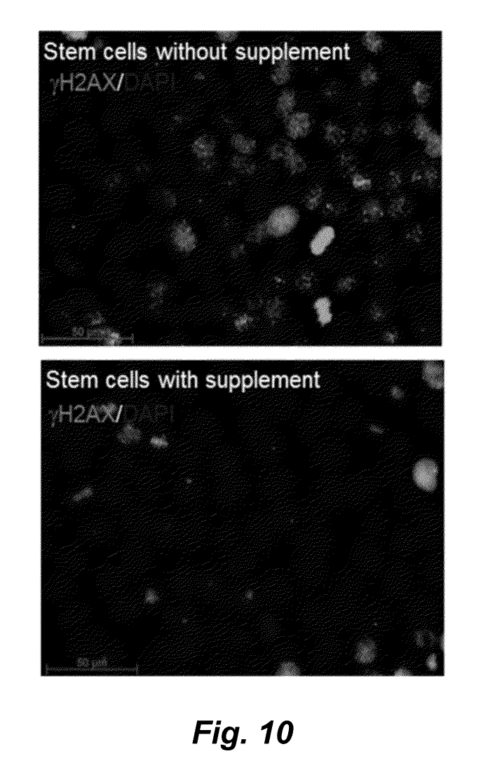

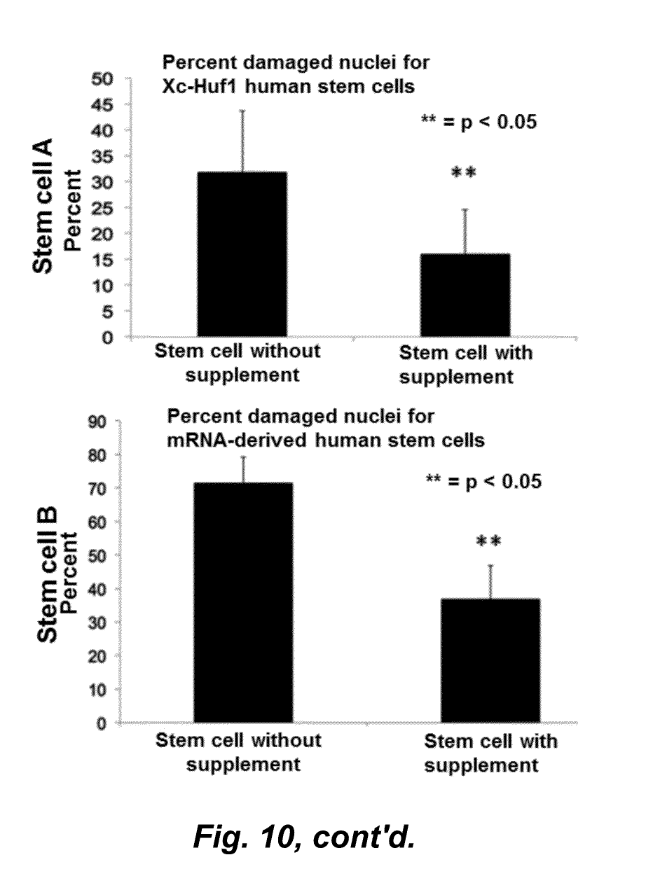

Data presented herein indicate that nucleoside supplementation can significantly reduce the incidence of genetic damage in human induced pluripotent stem cells, e.g., as measured by gammaH2A.X immunocytochemistry (see, e.g., FIG. 10) and it is believed that nucleoside supplementation can also reduce the incidence of genetic damage in a wide range of other human and non-human cell types.

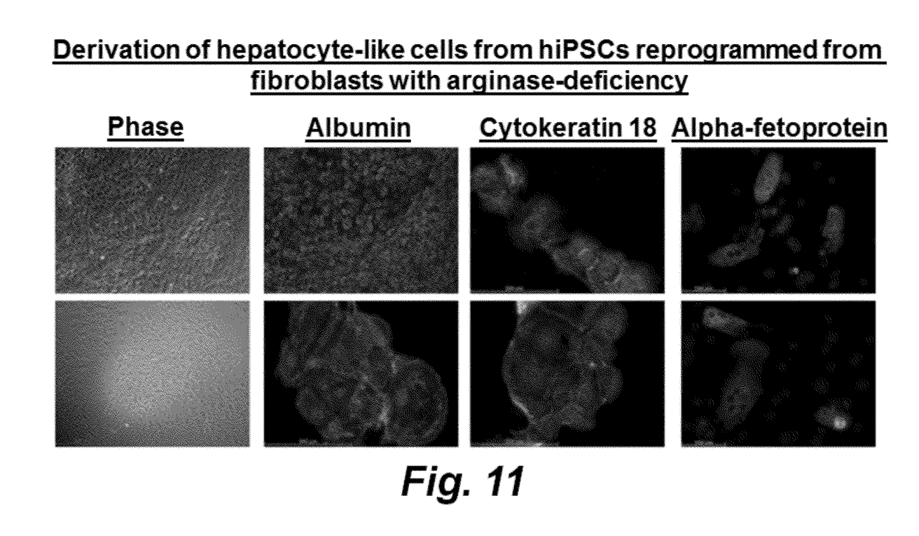

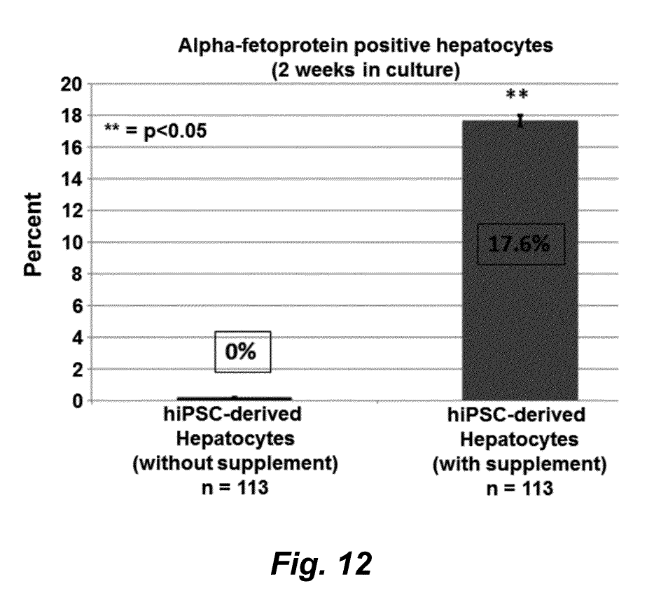

Data presented herein indicate that nucleoside supplementation can significantly augment the differentiation of hIPSCs into hepatocyte-like cells (see, e.g., FIGS. 11 and 12) and it is believed that nucleoside DC supplementation can also augment differentiation into a wide range of other human and non-human cell types.

Mammalian cells synthesize dNTPs via two pathways: the de novo pathway (DNP), which uses glucose and amino acids, and the nucleoside salvage pathway (NSP), which uses preformed dNs from the extracellular environment. Without being bound to a particular theory, it is believed that: 1) insufficient production of dNTP pools via the DNP is an important cause of replication stress, DNA damage and genomic instability in rapidly dividing hIPSCs (or other stem cells in culture) and 2) enabling the utilization by the hIPSC cells (or other stem cells) of the NSP by supplementing the culture medium with one or more deoxyribonucleotide substrates or precursors thereof will increase dNTP pools and will alleviate replication stress, DNA damage and genomic instability in stem cells (including iPSCs) in culture.

Similarly, it is believed that administration of a nucleoside cocktail transmission medium (NCT), e.g., a pharmaceutical or cosmetic formulation comprising one or more deoxynucleosides or precursors thereof as described herein will alleviate replication stress, DNA damage and genomic instability in stem cells and somatic cells (e.g., fibroblasts) in vivo.

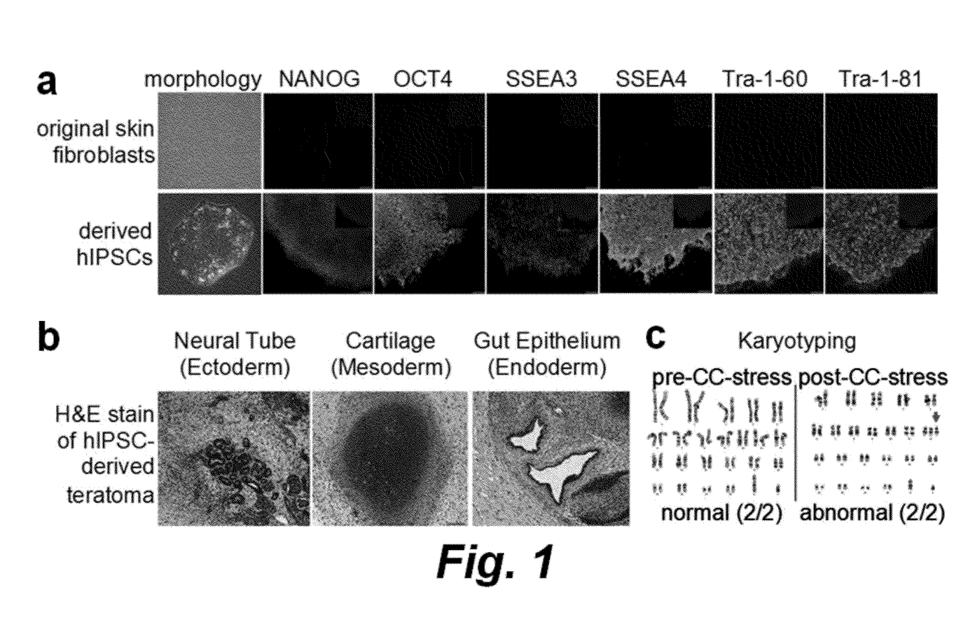

It was discovered that hIPSCs cultured under nucleoside-free conditions demonstrate dNTP deficiency (FIGS. 2 and 6), and high levels of genomic instability especially when placed under “clinical conversion” (CC)-based stress (FIG. 1). However, when standard hIPSC culture media was supplemented with different concentrations of dNs, it was found that 30 μM of each dN could increase hIPSC proliferation rate and genetic stability over 4-5 days (see, Example 1). It was also demonstrated that lower doses of dNs (e.g., 5 μM) will ensure genetic stability of hIPSCs (or other stem cells) over longer periods of time while also still significantly reducing the incidence of genomic damage as assayed via gammaH2A.X staining of double strand breaks.

The data presented herein show that a simple and cost-effective culture media supplementation approach that can be easily implemented by members of the stem cell biology and regenerative medicine field, can increase the clinical potential of personalized hIPSC-based cellular therapeutics.

The data presented herein show that a simple and cost-effective nucleoside supplementation approach that can be easily implemented by members of the cosmetic, regenerative and therapeutic fields to directly stimulate regeneration of human tissues (such as skin) Specifically, the direct topical application of a nucleoside cocktail (and/or other in vivo delivery mechanisms) will induce regeneration through the stimulation of proliferation of skin fibroblasts (FIG. 8), mesenchymal stem cells and/or other cells in the skin.

It is believed that nucleoside (e.g., dN) supplemented cells have improved genetic stability and pose significantly less risk of neoplasm formation following personalized stem cell-based (e.g., hIPSC-based) therapeutics, than stem cells derived, cultured and differentiated using the existing art (nucleoside-free (e.g., dN-free) culture media).

Accordingly, in various embodiments, culture media is provided that enhances the short term or long term genetic stability of stem cells (e.g., embryonic stem cells, adult stem cells, induced pluripotent stem cells, etc.) cultured in that medium. The culture media comprises essentially any culture medium utilized for the culture of stem cells (or other cells) where that medium is medium is supplemented with one or more deoxynucleoside or nucleoside (triphosphates) and/or precursors thereof

Similarly, the nucleoside cocktail transmission (NCT) medium comprises a pharmaceutical or cosmetic formulation (e.g., as described herein) containing one or more deoxynucleoside or nucleoside (triphosphates) and/or precursors thereof

### Culture Media.

In various embodiments the culture media comprise essentially any culture medium utilized for the culture of stem cells, where that medium is supplemented with one or more deoxynucleoside or nucleoside (triphosphates) and/or precursors thereof. In certain embodiments the culture medium is supplemented with two different deoxynucleoside and/or nucleoside (triphosphates) and/or precursors thereof. In certain embodiments the culture medium is supplemented with three different deoxynucleoside and/or nucleoside (triphosphates) and/or precursors thereof. In certain embodiments the culture medium is supplemented with four different deoxynucleoside and/or nucleoside (triphosphates) and/or precursors thereof. In certain embodiments the culture medium is supplemented with even more different deoxynucleoside and/or nucleoside (triphosphates) and/or precursors thereof

As indicated above, in various embodiments, the basal culture medium can be any culture medium capable of expanding, maintaining, or differentiating stem cells including IPSCs. In certain embodiments the basal medium may be manually prepared according to conventional methods. In certain embodiments the basal medium may be a commercially available medium or a mixture thereof. For example, the basal medium may be selected from the group consisting of DMEM (Dulbecco's Modified Eagle's Medium; GIBCO), MEM (Minimal Essential Medium; GIBCO), BME (Basal Medium Eagle; GIBCO), RPMI 1640 (GIBCO), DMEM/F-12 (Dulbecco's Modified Eagle's Medium: Nutrient Mixture F-12; GIBCO), DMEM/F-10 (Dulbecco's Modified Eagle's Medium: Nutrient Mixture F-10; GIBCO), α-MEM (α-Minimal essential Medium; GIBCO), G-MEM (Glasgow's Minimal Essential Medium; GIBCO), IMDM (Isocove's Modified Dulbecco's Medium; GIBCO), and KnockOut DMEM (GIBCO), essential 8 (E8) medium, and the like.

In various embodiments, the basal medium may contain one or more supplements, which includes, but not limited to, KnockOut Serum Replacement (GIBCO), KnockOut SR XenoFree (GIBCO), KnockOut SR XenoFree Growth Factor Cocktail (GIBCO), N2 supplement (GIBCO), B27 supplement (GIBCO), and so on. In certain embodiments the basal medium is a xenopathogen-free medium (i.e., xeno-free medium), in order to avoid any safety problem by materials derived from animal source. Typically such xeno-free medium does not include xenopathgen(s), such as bovine serum albumin, and recombinant proteins purified from animal cells.

As indicated above, such media can routinely be prepared in the laboratory, or can be commercially supplied. By way of non-limiting illustration, the composition of DMEM/F 12 is shown in Table 1.

TABLE 1
Composition of DMEM/F12 basal medium.

| | Molecular | Concentration | |
|---|---|---|---|
| Components | Weight | (mg/L) | mM |
| Amino acids: | | | |
| Glycine | 75 | 18.75 | 0.25 |
| L-Alanine | 89 | 4.45 | 0.05 |
| L-Arginine hydrochloride | 211 | 147.5 | 0.699052 |
| L-Asparagine-H2O | 150 | 7.5 | 0.05 |
| L-Aspartic acid | 133 | 6.65 | 0.05 |
| L-Cysteine hydrochloride-H2O | 176 | 17.56 | 0.099773 |
| L-Cystine 2HCl | 313 | 31.29 | 0.099968 |
| L-Glutamic Acid | 147 | 7.35 | 0.05 |
| L-Glutamine | 146 | 365 | 2.5 |
| L-Histidine hydrochloride-H2O | 210 | 31.48 | 0.149905 |
| L-Isoleucine | 131 | 54.47 | 0.415802 |
| L-Leucine | 131 | 59.05 | 0.450763 |
| L-Lysine hydrochloride | 183 | 91.25 | 0.498634 |
| L-Methionine | 149 | 17.24 | 0.115705 |
| L-Phenylalanine | 165 | 35.48 | 0.21503 |
| L-Proline | 115 | 17.25 | 0.15 |
| L-Serine | 105 | 26.25 | 0.25 |
| L-Threonine | 119 | 53.45 | 0.44916 |
| L-Tryptophan | 204 | 9.02 | 0.044216 |
| L-Tyrosine disodium salt | 261 | 55.79 | 0.213755 |
| dihydrate | | | |
| L-Valine | 117 | 52.85 | 0.451709 |
| Vitamins: | | | |
| Biotin | 244 | 0 | 0.000014 |
| Choline chloride | 140 | 8.98 | 0.064143 |
| D-Calcium pantothenate | 477 | 2.24 | 0.004696 |
| Folic Acid | 441 | 2.65 | 0.006009 |
| Niacinamide | 122 | 2.02 | 0.016557 |
| Pyridoxine hydrochloride | 206 | 2.01 | 0.009772 |
| Riboflavin | 376 | 0.22 | 0.000582 |
| Thiamine hydrochloride | 337 | 2.17 | 0.006439 |
| Vitamin B12 | 1,355 | 0.68 | 0.000502 |
| i-Inositol | 180 | 12.6 | 0.07 |
| Inorganic salts: | | | |
| Calcium Chloride (CaCl2) | 111 | 116.6 | 1.05045 |
| (anhyd.) | | | |
| Cupric sulfate (CuSO4-5H2O) | 250 | 0 | 0.000005 |
| Ferric Nitrate (Fe(NO3) | 404 | 0.05 | 0.000124 |
| 3″9H2O) | | | |
| Ferric sulfate (FeSO4-7H2O) | 278 | 0.42 | 0.0015 |
| Magnesium Chloride | 95 | 28.64 | 0.301474 |
| (anhydrous) | | | |
| Magnesium Sulfate (MgSO4) | 120 | 48.84 | 0.407 |
| (anhyd.) | | | |
| Potassium Chloride (KCl) | 75 | 311.8 | 4.157333 |
| Sodium Bicarbonate (NaHCO3) | 84 | 2,438 | 29.02381 |
| Sodium Chloride (NaCl) | 58 | 6,995.5 | 120.61207 |
| Sodium Phosphate dibasic | 142 | 71.02 | 0.500141 |
| (Na2HPO4) anhydrous | | | |
| Sodium Phosphate monobasic | 138 | 62.5 | 0.452899 |
| (NaH2PO4-H2O) | | | |
| Zinc sulfate (ZnSO4-7H2O) | 288 | 0.43 | 0.0015 |
| Other components: | | | |
| D-Glucose (Dextrose) | 180 | 3,151 | 17.505556 |
| Hypoxanthine Na | 159 | 2.39 | 0.015031 |
| Linoleic Acid | 280 | 0.04 | 0.00015 |
| Lipoic Acid | 206 | 0.1 | 0.00051 |
| Phenol Red | 376.4 | 8.1 | 0.02152 |
| Putrescine 2HCl | 161 | 0.08 | 0.000503 |
| Sodium Pyruvate | 110 | 55 | 0.5 |
| Thymidine | 242 | 0.36 | 0.001508 |

References:
1. Dulbecco, R. and Freeman, G., (1959) Plaque formation by the polyoma virus. Virology 8: 396.
2. Ham, R. G., (1965) Clonal growth of mammalian cells in a chemically defined synthetic medium. Proc. Natl. Acad. Sci., 53: 288.
3. Morton, H. J. (1970) A survey of commercially available tissue culture media. In vitro 6(2): 89.
4. Smith, J. D., Freeman, G., Vogt, M., et al., (1960) The nucleic acid of polyoma virus. Virology 12: 185.

The foregoing basal culture media are intended to be illustrative and non-limiting. One of skill will readily recognize that the supplements described herein can be used with virtually any culture media or incorporated into a pharmaceutical or cosmetic to form any of a number of NCT media.

### Nucleoside Cocktail Transmission (Delivery) Medium.

In certain embodiments the nucleoside cocktail transmission (NCT) medium comprises a delivery vehicle (e.g., a cosmetic formulation) containing one or more deoxynucleoside or nucleoside (triphosphates) and/or precursors thereof. The NCT medium is formulated to deliver the nucleoside(s) or nucleoside precursor(s) to a desired location in or on a mammal and to provide appropriate local concentration of the nucleoside or nucleoside precursor(s).

A “delivery vehicle” refers to, for example, a diluent, adjuvant, excipient, auxiliary agent or carrier with which one or more of the nucleosides and/or nucleoside precursors described herein is administered.

The NCTs can be formulated for subcutaneous, parenteral, topical, oral, nasal (or otherwise inhaled), or formulated for local administration, such as by aerosol or transdermally, e.g., to stimulate cellular proliferation of stem cells or other cells in vitro as well as to stimulate cellular proliferation and/or tissue rejuvenation/regeneration in vivo, and/or to improve cellular maintenance of introduced exogenous cells (e.g., stem cells), endogenous stem cells, somatic cells, and the like in a variety of contexts. The compositions can be administered in a variety of dosage/concentration forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

In certain embodiments, the NCTs are formulated for topical administration, subdermal administration, or intradermal administration. In certain formulation for administration to wound sites, including for example, acute wound sites, surgical sounds, burn sites, e.g., to promote healing and reduce scarring is contemplated.

In certain embodiments the NCTs are formulated with one or more pharmaceutical agents. Illustrative pharmaceutical agents include, but are not limited to, agents used for the treatment of wound sites, burn sites, acne, and other topical disfigurements (e.g., various kinds of dermal scarring). Illustrative agents include, but are not limited to for example, topical tamoxifen for dermal scarring, benzyol peroxide and antibiotics for acne, and the like.

Illustrative NCT vehicles, include cosmetic muds, herbal mixtures, fats (e.g., animal fats), emulsions, lotions, creams, gels, biologicals, gels, solutions, sprays, ointments, foams, mousses, liquids, suspensions, dispersions, aerosols, soaps, shampoos, conditioners, cleansers, and general cosmetic products. In certain embodiments, vehicles comprising agents for the reduction of wrinkles, and/or skin discoloration, and/or dermal fillers are contemplated.

Suitable vehicles to which the nucleoside(s) and/or nucleoside precursor(s) can be added include, but are not limited to, mud, herbal mixtures, animal fats, emulsions, lotions, creams, gels, biologicals, gels, solutions, sprays, ointments, foams, mousses, liquids, suspensions, dispersions, aerosols, soaps, shampoos, conditioners, cleansers, and general cosmetic products.

In certain embodiments, suitable vehicles include any carrier or vehicle commonly used as a base for creams, lotions, sprays, foams, gels, emulsions, lotions or paints for topical administration. Examples include emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol. Also contemplated are nonionic polyoxyethylene-polyoxypropylene copolymers, also referred to as poloxamers. One illustrative poloxamer is poloxamer 407, also known as

Pluronic F-127 (BASF). Additional carriers, include, but are not limited to, alginates, polyvinyl alcohol, hydrogels, including hydrogels that contain a cellulose derivative and/or polyacrylic acid; cellulose-based carrier, including hydroxyethyl cellulose, hydroxymethyl cellulose, carboxymethyl cellulose, hydroxypropylmethyl cellulose and mixtures thereof.

In certain embodiments the vehicle comprises a lotion. Lotions can contain finely powdered substances that are insoluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

In certain embodiments, the vehicle comprises a topical cream. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove. The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

In various embodiments the vehicle comprises an ointment. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

In certain embodiments the vehicle comprises a gel. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. ther additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

In certain embodiments the vehicle comprises a foam. Foams consist of an emulsion in combination with a gaseous propellant. The gaseous propellant consists primarily of hydro fluoroalkanes (HFAs). Suitable propellants include HFAs such as 1, 1,1,2-tetrafluoroethane (HFA 134a) and 1, 1, 1,2,3,3, 3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

It will be recognized that, in various embodiments the vehicles can contain additional ingredients, e.g., to enhance shelf-life, reduce biological contamination, improve wetting, maintain optimum pH, viscosity, maintain or improve color, smell, texture, and the like. Illustrative, additional ingredients include, but are not limited to excipients, diluents, emollients, surfactants, emulsifier, buffers, preservatives, penetration enhancer, scents, colorants, and the like.

Appropriate excipients are selected based on the type of formulation. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecyl sulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars, and starches.

“Diluents” may be included in the formulations to dissolve, disperse or otherwise incorporate the carrier. Examples of diluents include, but are not limited to, water, buffered aqueous solutions, organic hydrophilic diluents, such as monovalent alcohols, and low molecular weight glycols and polyols (e.g. propylene glycol, polypropylene glycol, glycerol, butylene glycol).

“Emollients” are an externally applied agent that softens or soothes skin and are generally known in the art and listed in compendia, such as the “Handbook of Pharmaceutical Excipients”, 4th Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

“Buffers” are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

“Preservatives” can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

“Penetration enhancers” are frequently used to promote transdermal delivery of drugs across the skin, in particular across the stratum corneum. A penetration enhancer may be added to enable the active agents to cross the barrier of the stratum corneum. Some penetration enhancers cause dermal irritation, dermal toxicity and dermal allergies. However, the more commonly used ones include urea, (carbonyldiamide), imidurea, N, N-diethylformamide, N-methy 1-2 -pyrrolidine, 1-dodecal-azacyclopheptane-2-one, calcium thioglycate, 2-pyyrolidine, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(l0 oxy ethylene ether), BRIJ® 78 (stearyl poly(20)oxyethylene ether), BRIJ® 96 (oleyl poly(l0)oxy ethylene ether), and BRIJ® 721 (stearyl poly (21) oxyethylene ether) (ICI Americas Inc. Corp.).

It will be recognized that the composition of the delivery vehicle will vary with the modality of administration, site of application, and the like. Methods of preparing pharmaceutical and cosmetic vehicles are well known to those of skill in the art (see, e.g., Remington's Pharmaceutical Sciences, 18th Ed. Mack Printing Company, 1990; Barel, Handbook of Cosmetic Science and Technology, 3rd Ed., CRC Press; and Rosen, Delivery System Handbook for Personal Care and Cosmetic Products: Technology, Applications and Formulations, Elsevier Science (2005)).

The foregoing formulations and administration methods are intended to be illustrative and not limiting. It will be appreciated that, using the teaching provided herein, other suitable formulations and modes of administration can be readily devised.

### Nucleoside Supplementation.

In various embodiments, the culture medium is supplemented with, or the NCT medium comprises one or more deoxynucleoside or nucleoside (triphosphates) and/or precursors thereof. Illustrative deoxyriboneucleoside triphosphates include, but need not be limited to deoxyadenosine triphosphate (dATP), deoxyguanosine triphosphate (dGTP), deoxycytidine triphosphate (dCTP), deoxythymidine triphosphate (dTTP), and deoxyuridine triphosphate (dUTP). Illustrative precursors of the dNTPs include, but are not limited to:

For deoxyadenosine triphosphate (dATP): deoxyadenosine diphosphate, deoxyadenosine monophosphate, deoxyadenosine, adenine, and the like. In some embodiments the precursors include one or more precursors selected from the group consisting of deoxyadenosine diphosphate, deoxyadenosine monophosphate, and deoxyadenosine, or any combination thereof

For deoxyguanosine triphosphate (dGTP): deoxyguanosine diphosphate, deoxyguanosine monophosphate, deoxyguanosine, guanine, and the like. In some embodiments the precursors include one or more precursors selected from the group consisting of deoxyguanosine diphosphate, deoxyguanosine monophosphate, and deoxyguanosine, or any combination thereof

For deoxycytidine triphosphate (dCTP): deoxycytidine diphosphate, deoxycytidine monophosphate, deoxycytidine, cytosine, and the like. In some embodiments the precursors include one or more precursors selected from the group consisting of deoxycytidine diphosphate, deoxycytidine monophosphate, and deoxycytidine, or any combination thereof

For deoxythymidine triphosphate (dTTP): deoxythymidine diphosphate, deoxythymidine monophosphate, deoxythymidine, thymine, and the like. In some embodiments the precursors include one or more precursors selected from the group consisting of deoxythymidine diphosphate, deoxythymidine monophosphate, and deoxythymidine, or any combination thereof

For deoxyuridine triphosphate deoxyuridine diphosphate, deoxyuridine monophosphate, deoxyuridine, uracil, and the like. In some embodiments the precursors include one or more precursors selected from the group consisting of deoxyuridine diphosphate, deoxyuridine monophosphate, and deoxyuridine, or any combination thereof

In various embodiments, the basal culture medium is additionally or alternatively supplemented with, or the NCT comprises one or more nucleoside triphosphates and/or precursors thereof. Illustrative nucleoside triphosphates include, but need not be limited to adenosine triphosphate (ATP), guanosine triphosphate (GTP), cytidine triphosphate (CTP), 5-methyluridine triphosphate (m5UTP), uridine triphosphate (UTP), and the like. In certain embodiments when ATP is present, at least one other nucleoside and/or deoxynucleoside triphosphate or precursor thereof is also present as a supplement. Illustrative precursors of the NTPs include, but are not limited to:

For adenosine triphosphate (ATP): adenosine diphosphate, adenosine monophosphate, adenosine, adenine, and the like, or any combination thereof. In some embodiments the precursors include one or more precursors selected from the group consisting of adenosine diphosphate, adenosine monophosphate, and adenosine.

For guanosine triphosphate (GTP): guanosine diphosphate, guanosine monophosphate, guanosine, guanine, and the like, or any combination thereof. In some embodiments the precursors include one or more precursors selected from the group consisting of guanosine diphosphate, guanosine monophosphate, and guanosine.

For cytidine triphosphate (CTP): cytidine diphosphate, cytidine monophosphate, cytidine, cytosine, and the like, or any combination thereof. In some embodiments the precursors include one or more precursors selected from the group consisting of cytidine diphosphate, cytidine monophosphate, and cytidine.

For 5-methyluridine triphosphate (m5UTP): 5-methyluridine diphosphate, 5-methyluridine monophosphate, 5-methyluridine, thymine, and the like, or any combination thereof. In some embodiments the precursors include one or more precursors selected from the group consisting of 5-methyluridine diphosphate, 5-methyluridine monophosphate, and 5-methyluridine.

For uridine triphosphate (UTP): uridine diphosphate, uridine monophosphate, uridine, uracil, and the like, or any combination thereof. In some embodiments the precursors include one or more precursors selected from the group consisting of uridine diphosphate, uridine monophosphate, and uridine.

In certain embodiments the precursor does not include the base alone.

In certain embodiments the culture medium is supplemented with, or the NCT medium comprises two different deoxynucleoside or nucleoside (triphosphates) and/or precursors thereof, or with three different deoxynucleoside or nucleoside (triphosphates) and/or precursors thereof, or with four (or more) different deoxynucleoside or nucleoside (triphosphates) and/or precursors. In certain embodiments, the culture medium is supplemented with, or the DCT medium comprises, at least, a purine and a pyramidine (or precursors thereof). In certain embodiments the culture medium is supplemented with, or the DCT medium comprises, at least, two purines (or precursors thereof). In certain embodiments the culture medium is supplemented with, or the NCT medium comprises, at least, two pyramidines (or precursors thereof). In certain embodiments the culture medium is supplemented with, or the NCT medium comprises, at least, two purines (or precursors thereof) and at least a pyramidine (or precursor thereof). In certain embodiments the culture medium is supplemented with, or the NCT medium comprises, at least, two pyramidines (or precursors thereof) and a purine (or precursor thereof). In certain embodiments the culture medium is supplemented with, or the NCT medium comprises, at least, two purines (or precursors thereof)and two pyramidines (or precursors thereof).

By way of illustration, in some embodiments the supplement (e.g., incorporated the culture medium or comprising the NCT medium) comprises or consists of one or more of the following:

1) dATP (or a precursor thereof);

2) dGTP (or a precursor thereof);

3) dCTP (or a precursor thereof);

4) dTTP (or a precursor thereof);

5) dUTP (or a precursor thereof) ;

6) dATP (or a precursor thereof) and dGTP (or a precursor thereof);

7) dATP (or a precursor thereof) and dCTP (or a precursor thereof);

8) dATP (or a precursor thereof) and dTTP (or a precursor thereof);

9) dATP (or a precursor thereof) and dUTP (or a precursor thereof);

10) dGTP (or a precursor thereof) and dCTP (or a precursor thereof);

11) dGTP (or a precursor thereof) and dTTP (or a precursor thereof);

12) dGTP (or a precursor thereof) and dUTP (or a precursor thereof);

13) dCTP (or a precursor thereof) and dTTP (or a precursor thereof);

14) dCTP (or a precursor thereof) and dUTP (or a precursor thereof);

15) dTTP (or a precursor thereof) and dUTP (or a precursor thereof);

16) dATP (or a precursor thereof), dGTP (or a precursor thereof), and dCTP (or a precursor thereof) ;

17) dATP (or a precursor thereof), dGTP (or a precursor thereof), and dTTP (or a precursor thereof);

18) dATP (or a precursor thereof), dCTP (or a precursor thereof), and dTTP (or a precursor thereof);

19) dGTP (or a precursor thereof), dCTP (or a precursor thereof), and dTTP (or a precursor thereof);

20) dATP (or a precursor thereof), dGTP (or a precursor thereof), and dCTP (or a precursor thereof) ;

21) dATP (or a precursor thereof), dGTP (or a precursor thereof), and dUTP (or a precursor thereof);

22) dATP (or a precursor thereof), dCTP (or a precursor thereof), and dUTP (or a precursor thereof);

23) dGTP (or a precursor thereof), dCTP (or a precursor thereof), and dUTP (or a precursor thereof);

24) dATP (or a precursor thereof), dGTP (or a precursor thereof), dCTP (or a precursor thereof), and dTTP (or a precursor thereof);

25) dATP (or a precursor thereof), dGTP (or a precursor thereof), dCTP (or a precursor thereof), and dUTP (or a precursor thereof);

It will be recognized that in any of the foregoing supplements, the deoxynucleoside can be a nucleoside or a precursor thereof. These supplements are intended to be illustrative and non-limiting.

Typically, the dNTPs and/or NTPs are present in the culture or NCT medium in an amount sufficient to improve the short term and/or long term genetic stability of stem cells as compared to the same cells cultured in the same culture medium or exposed to the NCT medium without supplementation by a nucleoside triphosphate or precursor thereof and/or to improve proliferation rate, and/or to improve viability.

In certain embodiments the NTPs and/or dNTPs or precursor(s) thereof supplementing the culture or DCT medium are each, independently, present at a concentration ranging from about lμM up to about 50 μM, or from about 1 μM to about 40 μM, or from about 1 μM up to about 35 μM, or from about 1 μM up to about 30 μM. In certain embodiments the NTPs and/or dNTPs or precursor(s) thereof supplementing the culture or DCT medium are each, independently, present at a concentration of about 50 μM or lower, or about 40 μM or lower, or about 35 μM or lower, or about 30 μM or lower or about 25 μM or lower, or about 20 μM or lower, or about 15 μM or lower, or about 10 μM or lower, or about 5 μM or lower (with the understanding that at least 0.1 μM of at least one nucleoside triphosphate or precursor thereof is present such that “lower” is not meant to be construed as an absence of every nucleoside triphosphate or precursor thereof).

In certain embodiments the NTPs and/or dNTPs or precursor(s) thereof supplementing the culture medium or comprising the NCT medium are each independently present at a concentration ranging from about 5 μM to about 30 μM.

In certain embodiments the NTPs and/or dNTPs or precursor(s) thereof supplementing the culture medium or comprising the NCT medium are each independently present at a concentration of about 1 μM, or about 2 μM, or about 3 μM, or about 4 μM, or about 5 μM, or about 6 μM, or about 7 μM, or about 8 μM, or about 9 μM, or about 10 μM, or about 11 μM, or about 12 μM, or about 13 μM, or about 14 μM, or about 15 μM, or about 16 μM, or about 17 μM, or about 18 μM, or about 19 μM, or about 20 μM, or about 21 μM, or about 22 μM, or about 23 μM, or about 24 μM, or about 25 μM, or about 26 μM, or about 27 μM, or about 28 μM, or about 29 μM, or about 30 μM, or about 31 μM, or about 32 μM, or about 33 μM, or about 34 μM, or about 35 μM, or about 36 μM, or about 37 μM, or about 38 μM, or about 39 μM, or about 40 μM, or about 41 μM, or about 42 μM, or about 43 μM, or about 44 μM, or about 45 μM, or about 46 μM, or about 47 μM, or about 48 μM, or about 49 μM, or about 50 μM.

In certain embodiments the NTPs and/or dNTPs or precursor(s) thereof supplementing the culture medium or comprising the NCT medium are each present at a concentration ranging from about μ1 μM, 1 μM, or about 2 μM, or about 3 μM, or about 4 μM, or about 5 μM up to about 50 μM, or up to about 40 μM, or up to about 30 μM, or up to about 25 μM, or up to about 20 μM, or up to about 15 μM.

In certain embodiments the total NTPs and/or dNTPs or precursor(s) thereof supplementing the culture medium or comprising the NCT medium is present at a concentration ranging from about 1 μM, or about 2 μM, or about 3 μM, or about 4 μM, or about 5 μM, or about 6 μM, or about 7 μM, or about 8 μM, or about 9 μM, or about 10 μM, or about 11 μM, or about 12 μM, or about 13 μM, or about 14 μM, or about 15 μM, or about 16 μM, or about 17 μM, or about 18 μM, or about 19 μM, or about 20 μM up to about 200 μM, or up to about 180 μM, or up to about 150 μM, or up to about 145 μM, or up to about 140 μM, or up to about 135 μM, or up to about 130 μM, or up to about 125 μM, or up to about 120 μM, or up to about 115 μM, or up to about 110 μM, or up to about 105 μM, or up to about 100 μM. In certain embodiments, a stem cell culture is also provided herein, where the stem cell culture comprises stem cells in a culture medium supplemented with one or more dNTPs and/or NTPs as described herein. In certain embodiments the stem cells can be in vivo and exposed to an NCT as described herein. In various embodiments, the stem cells may be embryonic stem cells or adult stem cells including, but not limited to neural stem cells, hepatic stem cells, hematopoietic stem cells, umbilical cord blood stem cells, epidermal stem cells, gastrointestinal stem cells, endothelial stem cells, muscle stem cells, mesenchymal stem cells, pancreatic stem cells, and the like. In certain embodiments the stem cells include induced pluripotent stem cells, especially human IPSCs. The stem cells (including IPSCs) can be non-human animal stem cells or human stem cells.

In certain embodiments the stem cells are induced pluripotent stem cells, especially human IPSCs. Illustrative stem cells include, but are not limited to, stem cells are selected from the group consisting of embryonic stem cells and adult stem cells, including, but not limited to neural stem cells, hepatic stem cells, hematopoietic stem cells, umbilical cord blood stem cells, epidermal stem cells, gastrointestinal stem cells, endothelial stem cells, muscle stem cells, mesenchymal stem cells, and pancreatic stem cells.

In certain embodiments, where the stem cells are IPSCs the IPSCs are reprogrammed from cells selected from the group consisting of fibroblasts, neural stem cells, stomach cells, liver cells, keratinocytes, melanocytes, amniotic cells, blood cells, β-cells, and adipose cells. In certain embodiments the IPSCs comprise cells reprogramed two or more factors selected from the group consisting of KLF4 (K), LIN28 (L), c-MYC (M), NANOG (N); OCT4 (O), SOX2 (S), and valproic acid (VPA). In certain embodiments the ISPCs comprises cells reprogramed using the four canonical Yamanaka factors KLF4 (K), c-MYC (M),OCT4 (O), and SOX2 (S).

Methods of reprogramming cells to produce IPSCs well known to those of skill in the art. In certain embodiments reprogramming can be accomplished using for example integrating vectors (e.g., lent viral vectors, inducible lentiviral vectors, and the like), excisable vectors (e.g., transposon vectors, loxP-flanked lentiviral vectors, and the like), non-integrating vectors (e.g., adenoviral vectors, plasmid vectors, etc.), DNA free vectors (e.g., Sendai virus, protein vectors, modified mRNA vectors, microRNA vectors, and the like). It is noted that a number of reprogramming kits are commercially available (e.g., (Epi5™ Episomal iPSC Reprogramming Kit and the CytoTune®-iPS Sendai Reprogramming Kit from Life Technologies, and the like).

In various embodiments, methods of reducing the genetic instability of induced stem cells (including pluripotent stem cells) are provided where the method comprises culturing the cells in a cell culture medium supplemented with NTPs and/or dNTPs and/or precursor(s) thereof as described herein or contacting the cells, e.g., in vivo, with a NCT medium comprising one or more NTPs and/or dNTPs and/or precursor(s) thereof as described herein..

In various embodiments a stem cell (e.g., an induced pluripotent stem cell) present in a cell culture medium supplemented with, or contacted with a NCT medium comprising, NTPs and/or dNTPs and/or precursor(s) thereof as described herein is provided.

Also provided are methods of providing autologous stem cell transfer. The methods typically involve providing stem cells isolated from a subject or IPSCs generated from a subject and expanding and/or culturing said stem cells or IPSCs in a cell culture medium or an NCT medium supplemented with NTPs and/or dNTPs and/or precursor(s) thereof as described herein.

In each of the embodiments described herein, the subject matter (i.e., cell culture, DCT medium, cells, methods, etc.) may be free of any NTPs and/or dNTPs and/or precursor(s) thereof that have been radiolabeled. Thus, in some embodiments, the NTPs and/or dNTPs and/or precursor(s) thereof used in such embodiments have not or are not linked or incorporating any radiolabel (such as, for example, ³H, ⁵¹Cr, or ³²P, and the like). Thus, the embodiments can be free of, for example, ³H-dTTP or ³H-TTP or any precursor thereof

The embodiments described herein are intended to be illustrative and non-limiting. Using the teachings provided herein, numerous variations of the compositions and methods described herein will be available to one of skill in the art.

### EXAMPLES

The following examples are offered to illustrate, but not to limit the claimed invention.

### Example 1

### Nucleoside Supplementation In hIPSC Culture Or In Nucleoside Cocktail

Transmission (NCT) Medium To Promote Genetic Stability, Enhance Cellular Function and Enhance Regeneration

### Significance

Human induced pluripotent stem cells (hIPSCs) have significant therapeutic potential (Byrne (2008) Human Molecular Genetics 17: R37-41). However, our findings (FIG. 1, Preliminary Data), and those of others (Martins-Taylor and Xu (2012) Stem Cells 30: 22-27; Lund et al. (2012) Nat. Rev. Genet., 13: 732-744), indicate that essentially all iPSC clones derived using current approaches acquire genomic instability during reprogramming and in vitro culture-induced stress. The issue of hIPSC genomic instability is one of the most important bottlenecks in advancing personalized iPSC-based regenerative therapies to the clinic (Martins-Taylor and Xu (2012) Stem Cells 30: 22-27; Lund et al. (2012) Nat. Rev. Genet., 13: 732-744; Byrne (2013) Gene Therapy and Regulation 7: 1230002), given the established link between genomic instability and an increased risk of malignant transformation.

The causes of genomic instability during hIPSC reprograming and culture remain unknown, and approaches to reduce and eventually eliminate the occurrence of this highly deleterious phenomenon have yet to be developed. In ongoing experiments, we find that deoxyribonucleotide triphosphate (dNTP) pools in rapidly dividing hIPSCs are significantly smaller in size than the dNTP pools of dermal fibroblasts, from which the hIPSCs are derived (FIG. 2). We also find that hIPSCs demonstrate significantly more double strand breaks (DSBs) than the fibroblasts of origin (FIGS. 3A and 3B). Our observations of decreased dNTP pools and increased DNA damage in hIPSCs may provide new mechanistic insight into the etiology of genomic instability of hIPSCs, and present a method for preventing this phenomenon. Thus, our data indicate that: 1) hIPSCs can utilize exogenous dNTP precursors added to the hIPSC culture medium in the form of a mixture of deoxyribonucleosides (dNs) and 2) that culture medium supplementation with dNs can both ameliorate the hIPSC dNTP pool deficiency and significantly reduce the genomic instability experienced by these cells (FIG. 4).

Hypotheses and Rationale.

Mammalian cells synthesize dNTPs via two pathways: the de novo pathway (DNP), which uses glucose and amino acids, and the nucleoside salvage pathway (NSP), which uses preformed dNs from the extracellular environment. Without being bound by a particular theory, we hypothesize that: 1) insufficient production of dNTP pools via the DNP is an important cause of replication stress, DNA damage and genomic instability in rapidly dividing hIPSCs and 2) enabling the utilization by the hIPSC cells of the NSP by supplementing the culture medium with a defined mixture of dN substrates of the NSP will increase dNTP pools and will alleviate replication stress, DNA damage and genomic instability.

Without being bound by a particular theory, it was hypothesized that: 1) insufficient production of dNTP pools from glucose and amino acids via the be novo pathway (DNP), is an important cause of replication stress, DNA damage and genomic instability in hIPSCs and 2) enabling the hIPSCs to also use a second dNTP biosynthetic pathway, the nucleoside salvage pathway (NSP) in addition to the DNP will alleviate replication stress, DNA damage, and genomic instability. In an illustrative embodiment, enabling the use of the NSP was accomplished by supplementing the hIPSC culture medium with a mixture of deoxyribonucleoside (dNs) substrates of the NSP.

In summary, a simple, effective, generally applicable, and cost-efficient solution to the problem of genomic instability is provided that is useful for hIPSCs produced using the current methodology. Specifically, it is shown that the occurrence of genomic instability in stem cells and hIPSCs can be prevented by enabling these cells to synthesize dNTPs via the NSP. This can be achieved by supplementing the culture media with an optimized mixture of dN substrates of the NSP

### Derivation and Characterization of Human Induced Pluripotent Stem Cells

We have reprogrammed adult human dermal fibroblasts (HDFs) into human induced pluripotent stem cells (hIPSCs) using either a LoxP-flanked polycistronic reprogramming vector (which was then removed from the genome using Cre recombinase (Sommer et al. (2010) Stem Cells 28: 64-74)), or by using a synthetic mRNA based approach (Warren et al. (2010) Cell Stem Cell, 7: 618-630). All reprogramming and subsequent culture were performed in standard hIPSC culture medium, which, according to the current standard practice, was not supplemented with any nucleosides (www.stemgent.com/products/show/69. NUTRISTEM™ XF/FF Culture Medium). Both transgene-free hIPSC lines demonstrated standard characterization parameters, as previously described (Byrne et al. (2009) PloS One 4: e7118). These parameters included embryonic stem cell (ESC)—like morphology, significant expression of the key pluripotency markers (NANOG, OCT4, SSEA3, SSEA4, Tra-1-60 and Tra-1-81), which were not detected in the original skin fibroblasts (FIG. 1, panel A), and differentiation into representatives of all three germ layers following teratoma formation in severe combined immunodeficiency (SCID) mice (FIG. 1, panel B). We then converted our transgene-free hIPSC lines from research-grade conditions (containing animal-derived epitopes: Matrigel substrate and Knockout Serum Replacement, KSR, containing culture medium) into putative clinical-grade animal epitope-free “xeno-free” defined conditions (CellStart substrate and an optimized xeno-free media, 50% mTeSRI, Stemcell Technologies, and 50% NutriStem, Stemgent), as previously described (Karumbayaram et al. (2012) Stem Cells Trans. Med. 1: 36-43). Both hIPSC lines were karyotyped, as we have previously described (Byrne et al. (2009) PloS One 4: e7118) both before and after “clinical-grade conversion” (CC).

Following CC-induced stress and expansion both lines went from being karyotypically normal (46XY) to possessing an extra chromosome 12 (FIG. 1, panel C). The observed genomic instability correlates closely with several published studies showing that hIPSCs are genetically unstable, particularly when placed under conditions of extrinsic stress (Nagaria et al. (2013) Biochimica et Biophysica Acta, 1830: 2345-2353), and preferentially duplicate chromosome 12 (Draper et al. (2004) Nat. Biotechnol. 22: 53-54). It has previously been suggested that all chromosomes in hIPSCs have the same initial incidence of chromosomal translocation and duplication, but that duplication of chromosome 12, when it occurs, results in a preferential expansion throughout the hIPSC population following extended in vitro culture through a selection process (Id.). It should be noted that we consider CC-based stress to be a useful model for longer term culture (>6-12 month) induced stress, which also tends to result in hIPSCs and hESCs accumulating karyotypic abnormalities, especially chromosome 12, as we observed following CC-based stress over a 2 month period.

Smaller dNTP Pools in hIPSCs vs. HDFs of Origin

We quantified deoxyribonucleotide triphosphate (dNTP) pools in the original HDFs and the early passage (none-CC-stressed) hIPSCs as described previously (Austin et al. (2012) J. Exp. Med., 209: 2215-2228). We observed a significant drop (between 32-52%) in levels of all four dNTPs after hIPSC reprogramming (FIG. 2).

Genes involved in dNTP Biosynthesis are Expressed at Similar Levels in Rapidly Dividing hIPSCs and in HDFs with a Slower Rate of Cell Division

An Affymetrix global transcriptional analysis on HDFs and derived early passage hIPSCs (performed as previously described (Awe et al. (2013) Stem Cell Res. & Theap., 4: 15)) showed significant upregulation of key pluripotency-specific markers; the expression of key fibroblast-specific markers was decreased following hIPSC reprogramming (Table 2). We also observed upregulation of genes previously linked to the induction of genomic instability (CCNE1, E2F2, E2F35). However, we did not observe any significant differences in the expression of key dNTP biosynthetic genes (Table 2). As hIPSCs have a faster average population doubling time (PDT) than HDFs (18 hrs vs. 48 hrs), these data support our hypothesis that the small dNTP pools in hIPSCs may result from 1) a faster rate of cell proliferation which increases the usage of dNTPs for DNA replication, and 2) an inability to upregulate the expression of genes involved in dNTP biosynthesis.

TABLE 2
Change in gene expression following hIPSC reprogramming.

| Gene | | | | |
|---|---|---|---|---|
| name | Gene ID | Type | Fold change | p-Value |
| POU5F1 | Hs.450254 | Pluripotency specific | 40 fold increase | 0.01 |
| NANOG | Hs.661360 | Pluripotency specific | 100 fold increase | 0.01 |
| SOX2 | Hs.518438 | Pluripotency specific | 30 fold increase | 0.004 |
| COL1A1 | Hs.172928 | Fibroblast specific | 20 fold decrease | 0.03 |
| COL3A1 | Hs.443625 | Fibroblast specific | 170 fold decrease | 0.001 |
| COL6A3 | Hs.233240 | Fibroblast specific | 40 fold decrease | 0.02 |
| CCNE1 | Hs.244723 | Genomic instability | 7 fold increase | 0.03 |
| E2F2 | Hs.194333 | Genomic instability | 8 fold increase | 0.02 |
| E2F3 | Hs.269408 | Genomic instability | 3 fold increase | 0.01 |
| dCK | Hs.709 | Nucleotide metabolism | No difference | N/A |
| TK1 | Hs.515122 | Nucleotide metabolism | No difference | N/A |
| UPP1 | Hs.488240 | Nucleotide metabolism | No difference | N/A |
| RM1 | Hs.445705 | Nucleotide metabolism | No difference | N/A |
| RRM2 | Hs.226390 | Nucleotide metabolism | No difference | N/A |

Elevated levels of DNA damage in hIPSCs vs. HDFs: dN supplementation reduces DNA damage in hIPSCs

We utilized gamma-histone 2A.X (yH2A.X) staining, as previously described (Bester et al. (2011) Cell, 145: 435-446), in order to estimate the number of double strand breaks (DSBs) in HDFs and early passage hIPSCs. Significantly more yH2A.X positive foci were detected in hIPSCs than in the HDFs of origin. We also observed significantly less yH2A.X positive foci following 4 days of dN supplementation (FIG. 3A, compare middle and lower panels). This finding provides additional support for the hypothesis that hIPSCs demonstrate higher levels of genomic instability than dermal fibroblasts and genomic instability could be prevented with dN supplementation of the cell culture medium. We also observed significant heterogeneity in the number of yH2A.X positive foci within the hIPSC population, supporting, our hypothesis that the incidence of genomic instability is non-uniform across the hIPSC population, and that a subpopulation of hIPSCs are more likely to incur additional genomic DNA damage, as assayed by yH2A.X staining, and thus represent the cells most susceptible to developing karyotypic abnormalities.

dN Supplementation Promotes hIPSC Genomic Stability Following CC-Based Stress

We cultured our hIPSCs in conditions with and without dNs in order to investigate whether dN supplementation could prevent genomic instability observed during CC-based stress. It should be noted that, while hIPSCs have been demonstrated to accumulate genomic damage during reprogramming and extended culture (Ben-David et al. (2010) Cell Cycle (Georgetown, Tex.) 9: 4603-4604), we observed the most extreme genomic damage (karyotypic abnormalities), in two independent hIPSC lines, when we stressed the cells by performing CC. This observation provides a rapid and consistent experimental assay to induce severe genomic damage over a relatively short period of time (2 months). Human iPSCs were first cultured on Matrigel, in a combination of mTeSRI (Stemcell Technologies) and NutriStem (Stemgent). They were then divided into two groups grown in the presence or absence of exogenously supplied dNs in the cell culture medium (30 μM of each dN). After one week of supplementation, we began the conversion process to xeno-free conditions, which entailed switching to CellStart as a basement membrane and NutriStem medium only. After two weeks of culture, genomic DNA (gDNA) was extracted for single nucleotide polymorphism (SNP)-based loss-of-heterozygosity (LOH) analysis, which was previously shown to be an accurate measure of DSB-based genomic instability (Bester et al. (2011) Cell, 145: 435-446). To determine levels of LOH between the two conditions (+/−dNs), analysis was performed using Affymetrix's SNP 6.0 array as previously described (Id.). Supplementation with dNs reduced the incidence of genomic instability, as assayed by LOH, to 20% of that observed in unsupplemented medium (FIG. 4). Together with the significant decrease in yH2A.X positive foci following 4 days of dN supplementation (FIGS. 3A and 3B), the reduction in LOH provides evidence in support of our hypothesis that dN supplementation reduces replication stress and the associated genomic instability in hIPSC reprogramming and culture.

It is important to note that previous research has correlated the incidence of DSB with LOH in human cancer cells (Id.), highlighting the value of SNP-based LOH measurements as a quantifiable method for assaying genomic instability. In our study of CC-stressed hIPSCs with and without dN supplementation, we observed a five-fold increase in genomic stability, as assayed by the reductions in LOH, following CC-induced stress. This can be observed in FIG. 4, where 12 LOH chromosomal loci were identified following CC-based stress. Five times as many LOH loci, which are indicative of genomic damage, were observed in the untreated hIPSCs (identified by green arrows), than were observed in the dN-treated hIPSCs (identified by the red arrow). This provides strong evidence in support of our hypothesis that dN supplementation can ameliorate CC-induced genomic instability in hIPSCs. However, it is important to note that half of the LOH loci (as identified by the blue arrows) were still unstable in both dN treated and untreated samples.

While efficacy has been demonstrated, it is believed the dN supplementation procedure can be further optimized. Data generated thus far support the hypothesis that dNTP pool deficiency plays a critical role in the genomic instability we and others have observed in hIPSCs following general culture and induced stress.

Moreover, our preliminary data also suggest a simple and generally applicable culture medium deoxyribonucleoside supplementation procedure to enhance the genomic stability of clinical grade hIPSCs and their derivatives.

In summary, we propose that establishing such an optimized media system capable of maintaining genetically stable stem cells and hIPSC and derivatives will be instrumental towards safely unlocking the future promise of personalized pluripotent stem cell-based therapeutics for patients.

### Example 2

### Obtaining Stable Stem dN Concentration

We have obtained multiple different STABLESTEM™ concentrations, one for short term culture (30 μM of each dN), one for long term culture (5 μM of each dN) as well as a suitable 30:30:5:5 cocktail that has proven robust in enhancing genomic stability, increasing cellular function and improving differentiation potential across multiple cell types (see, e.g. Table 3).

TABLE 3
dN concentrations.

| Culture | Dose of | 3-5 day cell | 3+ weeks cell |
|---|---|---|---|
| supplement | each dN | culture | culture |
| Short term | 30 μM | Good | Not good |
| Long term | 5 μM | Good | Good |

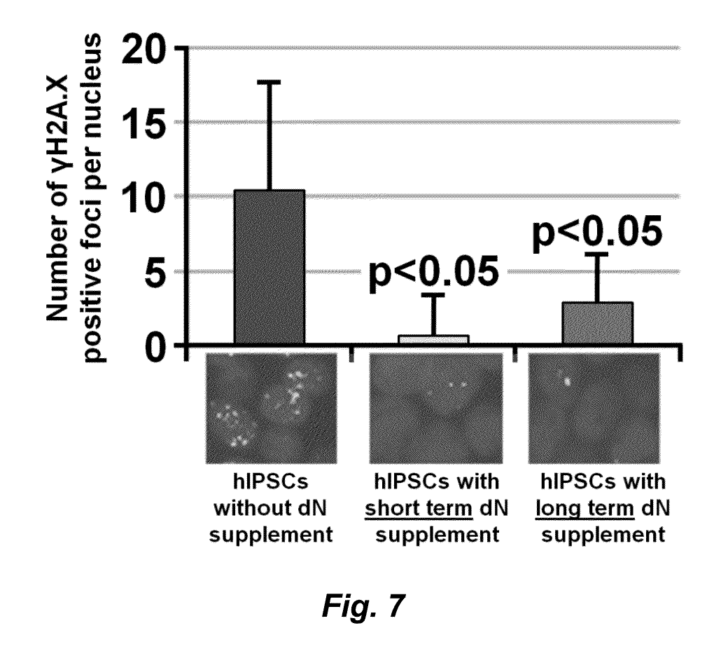

The long term STABLESTEM™ culture media supplement allows human iPS cells to proliferate for at least 3 weeks, while dramatically reducing the amount of genomic damage the cells are exposed to (FIG. 7); as measured by γH2A.X-positive double strand breaks. The causes of genomic instability during hIPSC reprograming and culture have been unclear. However, we recently discovered that deoxyribonucleotide triphosphate (dNTP) pools in rapidly dividing hIPSCs are significantly smaller in size than the dNTP pools of the skin cells from which the hIPSCs are derived. In addition, we discovered that supplementing the stem cell culture media with deoxyribonucleosides (dNs) can significantly rescue the dNTP pools (FIG. 6).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A cell culture medium for the culture of stem cells with improved genetic stability, said culture medium comprising:
a basal culture medium for stem cells, where said culture medium is supplemented with one or more nucleoside triphosphates or one or more precursors thereof.

2. A nucleoside cocktail transmission (NCT) medium for improving somatic or stem cell genetic stability said medium comprising:
a cosmetic or pharmaceutical delivery vehicle; and
one or more nucleoside triphosphates or precursors thereof.

3. The cell culture medium of claim 1, wherein said one or more nucleoside triphosphates are independently selected from the group consisting of deoxyadenosine triphosphate (dATP), deoxyguanosine triphosphate (dGTP), deoxycytidine triphosphate (NCTP), deoxythymidine triphosphate (dTTP) deoxyuridine triphosphate, adenosine triphosphate (ATP), guanosine triphosphate (GTP), cytidine triphosphate (CTP), 5-methyluridine triphosphate (m5UTP), and uridine triphosphate (UTP).

4. The nucleoside cocktail transmission medium of claim 2, wherein said one or more nucleoside triphosphates are independently selected from the group consisting of deoxyadenosine triphosphate (dATP), deoxyguanosine triphosphate (dGTP), deoxycytidine triphosphate (NCTP), deoxythymidine triphosphate (dTTP) deoxyuridine triphosphate, adenosine triphosphate (ATP), guanosine triphosphate (GTP), cytidine triphosphate (CTP), 5-methyluridine triphosphate (m5UTP), and uridine triphosphate (UTP).

5. (canceled)

6. The cell culture medium claim 1, wherein said culture medium is supplemented with one or more precursors of nucleoside triphosphates independently selected from the group consisting of deoxyadenosine triphosphate (dATP) precursor, deoxyguanosine triphosphate (dGTP) precursor, deoxycytidine triphosphate (NCTP) precursor, deoxythymidine triphosphate (dTTP) precursor, deoxyuridine triphosphate (dUTP) precursor, adenosine triphosphate (ATP) precursor, guanosine triphosphate (GTP) precursor, cytidine triphosphate (CTP) precursor, 5-methyluridine triphosphate (m5UTP) precursor, and uridine triphosphate (UTP) precursor.

7. The NCT medium of claim 2, wherein said NCT medium comprises one or more precursors of nucleoside triphosphates independently selected from the group consisting of deoxyadenosine triphosphate (dATP) precursor, deoxyguanosine triphosphate (dGTP) precursor, deoxycytidine triphosphate (NCTP) precursor, deoxythymidine triphosphate (dTTP) deoxyuridine triphosphate (dUTP) precursor, adenosine triphosphate (ATP) precursor, guanosine triphosphate (GTP) precursor, cytidine triphosphate (CTP) precursor, 5-methyluridine triphosphate (m5UTP) precursor, and uridine triphosphate (UTP) precursor.

8. The cell culture medium of claim 6, wherein said one or more precursors of nucleoside triphosphates comprise a nucleoside diphosphate or a nucleoside monophosphate.

9. The NCT medium of claim 7, wherein said one or more precursors of nucleoside triphosphates comprise a nucleoside diphosphate or a nucleoside monophosphate.

10-48. (canceled)

49. The nucleoside cocktail transmission (NCT) medium of claim 2, wherein said NCT medium comprises a vehicle formulated for administration via a route selected from the group consisting of subcutaneous administration, parenteral administration, topical administration, oral administration, nasal or inhalation administration, local administration such as by paint, aerosol, or transdermally.

50. (canceled)

51. The nucleoside cocktail transmission (NCT) medium of claim 49, wherein said medium is formulated in a vehicle selected from the group consisting of a mud, an herbal mixture, a fat, an emulsions, a lotion, a cream, a gel, a biological, a solution, a spray, an ointment, a foams, a mousses, a liquid, a suspensions, a dispersion, an aerosol, a soap, a shampoo, and a conditioner.

52. The NCT medium of claim 2, wherein said nucleoside triphosphate or precursor thereof is present at a concentration sufficient to improve the short term and/or long term genetic stability of stem cells as compared to the same cells in the same medium without supplementation by a nucleoside triphosphate or precursor thereof.

53. (canceled)

54. The cell culture medium of claim 1, wherein said nucleoside triphosphate or precursor thereof supplementing said culture medium, is present at a concentration sufficient to improve the short term and/or long term genetic stability of stem cells as compared to the same cells cultured in the same medium without supplementation by a nucleoside triphosphate or precursor thereof.

55-60. (canceled)

61. The cell culture medium of claim 1, wherein said cell culture medium is xenopathogen-free.

62. The cell culture medium of claim 1, wherein said cell culture medium, is without animal or human derived serum albumin.

63. The cell culture medium of claim 1, wherein the supplemented medium is selected from the group consisting of DMEM (Dulbecco's Modified Eagle's Medium), MEM (Minimal Essential Medium), BME (Basal Medium Eagle), RPMI 1640, DMEM/F-12 (Dulbecco's Modified Eagle's Medium: Nutrient Mixture F-12), DMEM/F-10 (Dulbecco's Modified Eagle's Medium: Nutrient Mixture F-10), α-MEM (α-Minimal essential Medium), G-MEM (Glasgow's Minimal Essential Medium), IMDM (Isocove's Modified Dulbecco's Medium), essential 8 (E8) medium, and KnockOut DMEM.

64-65. (canceled)

66. A somatic or stem cell culture or nucleoside cocktail transmission (NCT) culture, comprising:
cells in a basal culture medium for stem cells, where said culture medium is supplemented with one or more nucleoside triphosphates or one or more precursors thereof and/or
cells in a medium comprising a cosmetic or pharmaceutical delivery vehicle; and one or more nucleoside triphosphates or precursors thereof.

67. The cell culture or NCT culture of claim 66, wherein said cells are cells selected from the group consisting of somatic cells or stem cells.

68-75. (canceled)

76. A method of reducing genetic instability of stem cells said method comprising culturing said cells in a cell culture medium comprising a basal culture medium for stem cells, supplemented with one or more nucleoside triphosphates or one or more precursors thereof.

77. A method of performing autologous stem cell transfer, said method comprising isolating stem cells from a subject or generating IPSCs from said subject and expanding and/or culturing said stem cells or IPSCs in a cell culture medium comprising a basal culture medium for stem cells, supplemented with one or more nucleoside triphosphates or one or more precursors thereof.

78. A method of promoting regeneration and/or maintenance of tissues, said method comprising administering to a subject, an NCT medium comprising a cosmetic or pharmaceutical delivery vehicle and one or more nucleoside triphosphates or precursors thereof.

79-87. (canceled)
